(19) United States
(12) Patent Application Publication
(10) Pub. No.: US 2015/0322444 A1
(43) Pub. Date: Nov. 12, 2015

(54) ENHANCED ACCUMULATION OF CAROTENOIDS IN PLANTS

(71) Applicant: SYNGENTA LIMITED, Surrey (GB)

(72) Inventors: Caroline Rachel Drake, Bracknell (GB); Catherine Ann Shipton, Bracknell (GB); Jacqueline Ann Mary Paine, Bracknell (GB)

(73) Assignee: SYNGENTA LIMITED, Surrey (GB)

(21) Appl. No.: 14/806,226

(22) Filed: Jul. 22, 2015

Related U.S. Application Data
(62) Division of application No. 13/275,219, filed on Oct. 17, 2011, now abandoned.
Application No. 13/275,219 is a continuation of application No. 11/873,709, filed on Oct. 17, 2007, now abandoned.
Application No. 11/873,709 is a continuation of application No. 10/549,352, now abandoned.
(60) Provisional application No. 60/457,053, filed on Mar. 24, 2003.

(51) Int. Cl. C12N 15/82; C12N 9/10; C12N 9/02

(52) U.S. Cl. CPC C12N 15/825; C12N 9/0083; C12N 9/1085; C12Y 205/01032

(57) ABSTRACT

The present invention relates to polynucleotides and their use in methods of increasing the carotenoid content of seeds. In particular the invention provides a polynucleotide comprising: (a) a region which comprises as operably linked components (i) a promoter which provides for seed preferred expression; and (ii) a nucleotide sequence derived from a bacterium which sequence encodes a carotene desaturase; and (iii) a transcription termination region; and (b) a further region which comprises as operably linked components (i) a promoter which provides for seed preferred expression; and (ii) a nucleotide sequence encoding a phytoene synthase which sequence is derived from maize (Zea sp.) or rice (Orzya sp.); and (iii) a transcription termination region. The disclosed polynucleotides are particularly suitable for use in production of rice seed which comprise high amounts of coloured carotenoids.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 70237-US-REG-D-NAT-1_SeqListC2.txt, a creation date of Oct. 14, 2011 and a size of 116 KB. The sequence listing filed via EFS-Web is part of the specification and is hereby incorporated in its entirety by reference herein.

The present invention relates inter alia, to recombinant DNA technology. More specifically the invention relates to the provision of improved polynucleotides which provide for an enhanced accumulation of carotenoids in plants and in particular in the seeds of said plants. The invention also provides plant material, plants and seeds which comprise the polynucleotides, in particular rice plant material, rice plants and rice seeds.

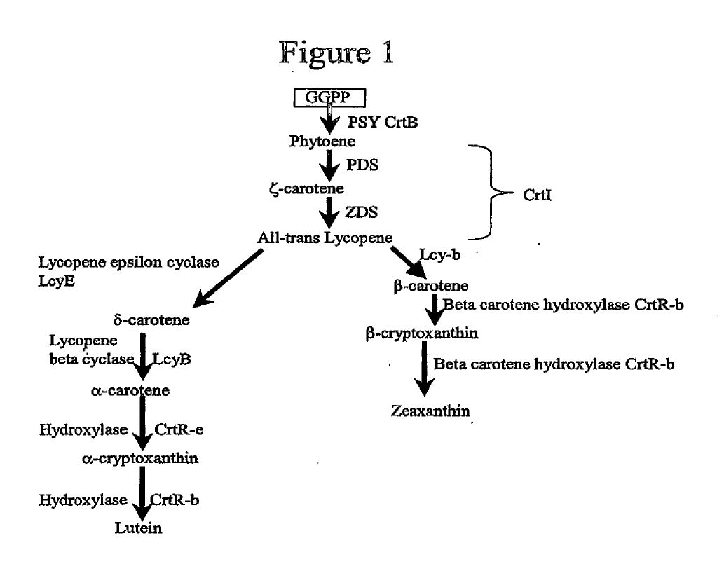

Carotenoids are 40-carbon (C₄₀) isoprenoids formed by condensation of eight isoprene units derived from the biosynthetic precursor isopentenyl diphosphate. By nomenclature, carotenoids basically fall into two classes, namely, carotenes and xanthophylls. Their essential function in plants is to protect against photo-oxidative damage in the photosynthetic apparatus of plastids. In addition to this, carotenoids participate in light harvesting during photosynthesis and represent integral components of photosynthetic reaction centres. Carotenoids are the direct precursors of the phytohormone abscisic acid. A part of the carotenoid biosynthesis pathway is shown in FIG. 1.

Carotenoids with provitamin A activity are essential components of the human diet. Additionally, there is compelling evidence suggesting that a diet rich in carotenoids can prevent a number of serious medical conditions from developing, including certain cancers (especially lung and prostate), macular degeneration, cateract and cardiovascular disease. Carotenoids have also been reported to have immunomodulatory effects, such as the reduction in UV-induced immunosuppression. Carotenoids are able to act as efficient quenchers of harmful reactive oxygen species such as singlet oxygen and thereby have antioxidant properties.

With the population of the world increasing, there remains a need for the production of foods which are high in nutrition, healthy, tasty and visually appealing.

The general insertion of genes involved in the carotenoid biosynthesis pathway into plants is disclosed in WO00/53768. U.S. Pat. No. 6,429,356 describes methods for the production of plants and seeds having altered fatty acid, tocopherol and carotenoid compositions via insertion of a crtB gene. The present invention provides, inter alia, improved polynucleotides which when inserted into plant material provide for a surprisingly high accumulation of carotenoids in at least the seeds of plants derived from said material. More specifically, the present invention provides particular combinations of nucleotide sequences which, when expressed in plant material provide for a surprisingly high accumulation of carotenoids in at least the seeds of plants derived from said material.

According to the present invention there is provided a polynucleotide comprising: (a) a region which comprises as operably linked components (i) a promoter which provides for seed preferred expression; and (ii) a nucleotide sequence derived from a bacterium which sequence encodes a carotene desaturase; and (iii) a transcription termination region; and (b) a further region which comprises as operably linked components (i) a promoter which provides for seed preferred expression; and (ii) a nucleotide sequence encoding a phytoene synthase which sequence is derived from maize (Zea sp.) or rice (Orzya sp.); and (iii) a transcription termination region.

The invention further provides a polynucleotide comprising: (a) a region which comprises as operably linked components (i) a promoter which provides for seed preferred expression; and (ii) a nucleotide sequence derived from a bacterium which sequence encodes a carotene desaturase; and (iii) a transcription termination region; and (b) a further region which comprises as operably linked components (i) a promoter which provides for seed preferred expression; and (ii) a nucleotide sequence encoding a phytoene synthase which sequence is derived from tomato (Lycopersicon sp.) or pepper (Capsicum sp.) or a bacterium; and (iii) a transcription termination region.

In a further embodiment said carotene desaturase is derivable from Streptomyces; Staphylococcus; Synechocystis; Rhodobacter; Paracoccus; Erwinia; and Xanthophyllomyces. In a further embodiment said carotene desaturase is a phytoene desaturase. In a further embodiment when said phytoene synthase is derivable from a bacterium, it is derivable from Streptomyces; Staphylococcus; Synechocystis; Rhodobacter; Paracoccus; Erwinia; and Xanthophyllomyces. In a further embodiment of the invention said phytoene synthase is obtainable from Zea mays or Orzya sativa. In a still further embodiment of the invention said phytoene synthase is obtainable from Lycopersicon esculentum or Capsicum anuum.

In a further embodiment said phytoene synthase is obtained from Zea mays or Orzya sativa. In a still further embodiment said phytoene synthase comprises, is comprised by or consists of the sequence selected from the group depicted as SEQ ID NOS: 10; 11; 12; and 13. In a further embodiment said phytoene synthase comprises, is comprised by or consists of a sequence which encodes the protein depicted as SEQ ID NO 14. Alternative phytoene synthase encoding sequences may also be available from databases known to the person skilled in the art. For example, the sequences depicted in the EMBL Database under Accession Numbers—AY024351, AK073290, AK108154 and AY078162.

In a still further embodiment said phytoene synthase is derived, obtainable or obtained from Lycopersicon esculentum or Capsicum anuum. In a still further embodiment said phytoene synthase comprises, is comprised by or consists of SEQ ID NO: 15 or SEQ ID NO: 16.

In a still further embodiment said phytoene synthase is derived, obtainable or obtained from the CrtB gene from bacteria. In a particular embodiment said crtB comprises, is comprised by or consists of the crtB sequence depicted in Shewmaker et al. (1999) Plant J. 20:401-12. In a further embodiment of the invention the sequence which encodes a carotene desaturase and/or the sequence encoding a phytoene synthase from a bacteria is derived from Erwinia sp., more specifically, Erwinia uredovora. In a still further embodiment the carotene desaturase is derived from the CrtI gene from Erwinia uredovora. In a still further embodiment the carotene desaturase is the CrtI gene from Erwinia uredovora. In a still further embodiment the carotene desaturase comprises, is comprised by or consists of the sequence depicted as SEQ ID NO: 18 or SEQ ID NO: 19.

The present invention further provides a polynucleotide as described above wherein said promoter is selected from the Glutelin 1 promoter and the Prolamin promoter and said transcription termination region is selected from the Nos; CaMV 35S and PotP1-II transcription termination regions. Said Glutelin 1 and Prolamin promoters may be isolated from rice. In a particular embodiment said promoter comprises the sequence depicted as SEQ ID NO: 20 or 21. Further promoters include the promoter derived from the napin gene from Brassica napus and other promoters which are derived from genes normally expressed in the endosperm of the seed. Further transcription termination regions include the terminator region of a gene of alpha-tubulin (EP-A 652,286). It is equally possible to use, in association with the promoter regulation sequence, other regulation sequences which are situated between the promoter and the sequence encoding the protein, such as transcriptional or translational enhancers, for example, tobacco etch virus (TEV) translation activator described in International Patent application, PCT publication number WO87/07644.

The present invention still further provides a polynucleotide as described above wherein the sequence which encodes carotene desaturase and the sequence which encodes phytoene synthase further comprises a plastid targeting sequence. In a particular embodiment the plastid targeting sequence is derived from the ribulose bisphosphate carboxylase small-subunit (RUBISCO Ssu) from Pisum sativum. In a further embodiment said RUBISCO Ssu plastid targeting sequence is located 5′ to the translational start point of said carotene desaturase gene derived from CrtI. In a still further embodiment said RUBISCO Ssu plastid targeting sequence is located 5′ to the translational start point of said phytoene synthase gene derived from CrtB. In a further embodiment said plastid targeting sequence is heterologous with respect to said phytoene synthase and/or said carotene desaturase. In a still further embodiment said plastid targeting sequence is autologous with respect to said phytoene synthase and/or said carotene desaturase. By “heterologous” is meant from a different source, and correspondingly “autologous” means from the same source—but at a gene rather than organism or tissue level. In a still further embodiment the plastid targeting sequence associated with the carotene desaturase is heterologous therewith and the plastid targeting sequence associated with the phytoene synthase is autologous therewith. In a still further embodiment the plastid targeting sequence provides for the accumulation of carotenoids in the amyloplast of the seed.

The present invention still further provides a polynucleotide as described above wherein the sequence which encodes said carotene desaturase and/or the sequence which encodes said phytoene synthase further comprises an intron. In a particular embodiment said intron region is located between the promoter region and the region encoding the carotene desaturase/phytoene synthase. In a further embodiment said intron region is located between the promoter region and the plastid targeting sequence. In a still further embodiment said intron region is located upstream of said carotene desaturase and/or said phytoene synthase encoding sequence(s). In a still further embodiment said intron is derived from the intron of the first gene from the catalase gene of the castor bean plant. In a still further embodiment said intron is from the first intron of the catalase gene from the castor bean plant. In a still further embodiment said intron comprises the sequence depicted as SEQ ID NO: 22. In a still further embodiment the intron is the intron of the maize polyubiquitin gene.

The present invention further provides a polynucleotide as described above wherein said sequence encoding carotene desaturase is located 5′ to said sequence encoding phytoene synthase.

The present invention further provides a polynucleotide as described above wherein said sequence encoding phytoene synthase is located 5′ to said sequence encoding carotene desaturase.

The present invention still further provides a polynucleotide as described above which comprises the sequence selected from the group depicted as SEQ ID NOS: 1; 2; 3; 4; 5; and 6. In a particular embodiment of the invention the polynucleotide consists of a sequence selected from the group depicted as SEQ ID NO: 1; 2; 3; 4; 5 and 6. In a further embodiment of the invention the polynucleotide comprises or consists of SEQ ID NO: 1. In a still further embodiment of the invention the polynucleotide comprises or consists of SEQ ID NO: 2. In a still further embodiment of the invention the polynucleotide comprises or consists of SEQ ID NO: 3. In a still further embodiment of the invention the polynucleotide comprises or consists of SEQ ID NO: 4. In a still further embodiment of the invention the polynucleotide comprises or consists of SEQ ID NO: 6. The present invention still further provides a polynucleotide as described above which comprises or consists of a sequence selected from the group depicted as SEQ ID NOS: 7; 8; and 9.

The present invention still further provides a polynucleotide sequence which is the complement of one which hybridises to a polynucleotide as described in the preceding paragraph at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still comprises a region encoding a carotene desaturase and a further region encoding a phytoene synthase and when said polynucleotide sequence is inserted into plant material the seed of a plant regenerated from said material produce an increased amount of carotenoids when compared to a control like-seed. The skilled person may alternatively select the following hybridisation conditions, viz., hybridisation at a temperature of between 60° C. and 65° C. in 0.3 strength citrate buffered saline containing 0.1% SDS followed by rinsing at the same temperature with 0.3 strength citrate buffered saline containing 0.1% SDS followed by confirmation that when the polynucleotide sequence so identified is inserted into plant material the seed of a plant regenerated from said material produce an increased amount of carotenoids when compared to a control like-seed. The person skilled in the art may also select further hybridisation conditions that are equally understood to be “high stringency” conditions. In a particular embodiment of the present invention when said polynucleotide sequence is inserted into plant material, the seed of a plant regenerated from said material produces at least a sixty fold increase in carotenoids when compared to a control like-seed. In a further embodiment of the invention when said polynucleotide sequence is inserted into plant material, the seed of a plant regenerated from said material produces at least a one hundred fold increase in carotenoids when compared to a control like-seed. In a still further embodiment of the invention when said polynucleotide sequence is inserted into plant material, the seed of a plant regenerated from said material produces at least a one hundred and fifty fold increase in carotenoids when compared to a control like-seed. In a still further embodiment of the invention when said polynucleotide sequence is inserted into plant material, the seed of a plant regenerated from said material produces at least a two hundred fold increase in carotenoids when compared to a control like-seed. In a still further embodiment of the invention when said polynucleotide sequence is inserted into plant material, the seed of a plant regenerated from said material produces at least a two hundred and fifty fold increase in carotenoids when compared to a control like-seed. In a still further embodiment of the invention when said polynucleotide sequence is inserted into plant material, the seed of a plant regenerated from said material produces at least a three hundred fold increase in carotenoids when compared to a control like-seed. In a still further embodiment of the invention when said polynucleotide sequence is inserted into plant material, the seed of a plant regenerated from said material produces at least a three hundred and fifty fold increase in carotenoids when compared to a control like-seed. In a still further embodiment of the invention when said polynucleotide sequence is inserted into plant material, the seed of a plant regenerated from said material produces at least a four hundred fold increase in carotenoids when compared to a control like-seed. In a still further embodiment of the invention when said polynucleotide sequence is inserted into plant material, the seed of a plant regenerated from said material produces at least a five hundred fold increase in carotenoids when compared to a control like-seed.

The term control like-seed relates to seeds which are substantially similar to those according to the invention but which control like-seed does not contain the polynucleotides or polynucleotide sequences according to the invention. Typically, a control like-seed will comprise a seed of the same or similar plant species which control like-seed has not been transformed. The increased carotenoid content of the seeds comprising the polynucleotides or polynucleotide sequences according to the invention may also be demonstrated via comparison of said seeds with seeds that comprise the TDNA depicted in Plasmid A of FIG. 4 of WO00/53768, wherein the phytoene synthase (psy) is from daffodil (Narcissus pseudonarcissus). Typically, such a comparison would be made when the seed to be compared are of the same or a substantially similar plant species. In a particular embodiment the seed comprising the polynucleotides or polynucleotide sequences according to the invention contain at least three times the amount of carotenoids when compared to a seed that comprise the TDNA depicted in Plasmid A of FIG. 4 of WO00/53768, wherein the phytoene synthase (psy) is from daffodil (Narcissus pseudonarcissus). In a still further embodiment the seed comprising the polynucleotides or polynucleotide sequences according to the invention contains at least four times, or at least five times, or at least six times, or at least seven times, or at least eight times or at least nine times or at least ten times or at least fifteen times or at least twenty times or at least thirty times, or at least forty times or at least fifty times the amount of carotenoids when compared to a seed that comprise the TDNA depicted in Plasmid A of FIG. 4 of WO00/53768, wherein the phytoene synthase (psy) is from daffodil (Narcissus pseudonarcissus).

The present invention still further provides a polynucleotide sequence as described above wherein when said polynucleotide sequence is inserted into plant material, the seed of a plant regenerated from said material produces carotenoids at a level of at least 3 μg/g of endosperm of said seed. In a further embodiment when said polynucleotide sequence as described above is inserted into plant material, the seed of a plant regenerated from said material produces carotenoids at a level of at least 4 μg/g of endosperm of said seed. In a further embodiment when said polynucleotide sequence as described above is inserted into plant material, the seed of a plant regenerated from said material produces carotenoids at a level of at least 5 μg/g of endosperm of said seed. In a still further embodiment when said polynucleotide sequence as described above is inserted into plant material, the seed of a plant regenerated from said material produces carotenoids at a level of at least 6 μg/g of endosperm of said seed In a still further embodiment when said polynucleotide sequence as described above is inserted into plant material, the seed of a plant regenerated from said material produces carotenoids at a level of at least 7 μg/g of endosperm of said seed. In a still further embodiment when said polynucleotide sequence as described above is inserted into plant material, the seed of a plant regenerated from said material produces carotenoids at a level of at least 8 μg/g of endosperm of said seed. In a still further embodiment when said polynucleotide sequence as described above is inserted into plant material, the seed of a plant regenerated from said material produces carotenoids at a level of at least 9 μg/g of endosperm of said seed. In a still further embodiment when said polynucleotide sequence as described above is inserted into plant material, the seed of a plant regenerated from said material produces carotenoids at a level of at least 10 μg/g of endosperm of said seed. In a still further embodiment when said polynucleotide sequence as described above is inserted into plant material, the seed of a plant regenerated from said material produces carotenoids at a level of at least 11 μg/g of endosperm of said seed. In a still further embodiment when said polynucleotide sequence as described above is inserted into plant material, the seed of a plant regenerated from said material produces carotenoids at a level of at least 12 μg/g of endosperm of said seed. In a still further embodiment when said polynucleotide sequence as described above is inserted into plant material, the seed of a plant regenerated from said material produces carotenoids at a level of at least 13 μg/g of endosperm of said seed. In a still further embodiment when said polynucleotide sequence as described above is inserted into plant material, the seed of a plant regenerated from said material produces carotenoids at a level of at least 14 μg/g of endosperm of said seed.

In a still further embodiment when said polynucleotide sequence as described above is inserted into plant material, the seed of a plant regenerated from said material produces carotenoids at a level of at least 15 μg/g of endosperm of said seed. In a still further embodiment when said polynucleotide sequence as described above is inserted into plant material, the seed of a plant regenerated from said material produces carotenoids at a level of at least 20 μg/g of endosperm of said seed. In a still further embodiment when said polynucleotide sequence as described above is inserted into plant material, the seed of a plant regenerated from said material produces carotenoids at a level of at least 25 μg/g of endosperm of said seed. In a still further embodiment when said polynucleotide sequence as described above is inserted into plant material, the seed of a plant regenerated from said material produces carotenoids at a level of at least 30 μg/g of endosperm of said seed. In a still further embodiment when said polynucleotide sequence as described above is inserted into plant material, the seed of a plant regenerated from said material produces carotenoids at a level of at least 35 μg/g of endosperm of said seed. In a still further embodiment when said polynucleotide sequence as described above is inserted into plant material, the seed of a plant regenerated from said material produces carotenoids at a level of at least 40 μg/g of endosperm of said seed. In a still further embodiment when said polynucleotide sequence as described above is inserted into plant material, the seed of a plant regenerated from said material produces carotenoids at a level of at least 45 μg/g of endosperm of said seed. In a still further embodiment when said polynucleotide sequence as described above is inserted into plant material, the seed of a plant regenerated from said material produces carotenoids at a level of at least 50 μg/g of endosperm of said seed. In a still further embodiment when said polynucleotide sequence as described above is inserted into plant material, the seed of a plant regenerated from said material produces carotenoids at a level of at least 55 μg/g of endosperm of said seed. In a still further embodiment when said polynucleotide sequence as described above is inserted into plant material, the seed of a plant regenerated from said material produces carotenoids at a level of at least 60 μg/g of endosperm of said seed. In a still further embodiment when said polynucleotide sequence as described above is inserted into plant material, the seed of a plant regenerated from said material produces carotenoids at a level of at least 65 μg/g of endosperm of said seed. In a particular embodiment the amount of carotenoids is calculated as μg/g of dry weight of endosperm of said seed.

The present invention still further provides a polynucleotide sequence which is the complement of one which hybridises to a polynucleotide which consists of a sequence selected from the group depicted as SEQ ID NOS: 1; 2; 3; 4; 5; and 6 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still comprises a region encoding a carotene desaturase and a further region encoding a phytoene synthase and when said polynucleotide sequence is inserted into plant material the seed of a plant regenerated from said material produce carotenoids amounting to at least 80% of the carotenoid content of a seed which comprises a polynucleotide selected from the group depicted as SEQ ID NOS: 1; 2; 3; 4; 5 and 6. In a further embodiment, when said polynucleotide sequence is inserted into plant material, the seed of a plant regenerated from said material produce carotenoids amounting to at least 85% of the carotenoid content of a seed which comprises a polynucleotide selected from the group depicted as SEQ ID NOS: 1; 2; 3; 4; 5 and 6. In a still further embodiment when said polynucleotide sequence is inserted into plant material, the seed of a plant regenerated from said material produce carotenoids amounting to at least 90% of the carotenoid content of a seed which comprises a polynucleotide selected from the group depicted as SEQ ID NOS: 1; 2; 3; 4; 5; and 6. In a still further embodiment when said polynucleotide sequence is inserted into plant material, the seed of a plant regenerated from said material produce carotenoids amounting to at least 95% of the carotenoid content of a seed which comprises a polynucleotide selected from the group depicted as SEQ ID NOS: 1; 2; 3; 4; 5 and 6. In a still further embodiment when said polynucleotide sequence is inserted into plant material, the seed of a plant regenerated from said material produce carotenoids amounting to at least 100% of the carotenoid content of a seed which comprises a polynucleotide selected from the group depicted as SEQ ID NOS: 1; 2; 3; 4; 5 and 6. In a particular embodiment the polynucleotide sequence provides for a percentage of the carotenoid content of seed as described above wherein the seed with which the comparison is made comprises the polynucleotide depicted as SEQ ID NO: 1. In a particular embodiment the polynucleotide sequence provides for a percentage of the carotenoid content of seed as described above wherein the seed with which the comparison is made comprises the polynucleotide depicted as SEQ ID NO: 2. In a particular embodiment the polynucleotide sequence provides for a percentage of the carotenoid content of seed as described above wherein the seed with which the comparison is made comprises the polynucleotide depicted as SEQ ID NO: 3. In a particular embodiment the polynucleotide sequence provides for a percentage of the carotenoid content of seed as described above wherein the seed with which the comparison is made comprises the polynucleotide depicted as SEQ ID NO: 4. In a particular embodiment the polynucleotide sequence provides for a percentage of the carotenoid content of seed as described above wherein the seed with which the comparison is made comprises the polynucleotide depicted as SEQ ID NO: 6.

It is preferred that when the carotenoid content of the seed comprising said polynucleotide sequence is compared with the seed comprising the polynucleotide selected from the group depicted as SEQ ID NOS: 1; 2; 3; 4; 5 and 6, the seed are from plants of substantially the same species. It is further preferred that when the carotenoid content of the seed comprising said polynucleotide sequence is compared with the seed comprising the polynucleotide selected from the group depicted as SEQ ID NOS: 1; 2; 3; 4; 5 and 6, the seed are from plants which are substantially genetically identical save for the presence of said polynucleotide or said polynucleotide sequence. It is further preferred that when the carotenoid content of the seed comprising said polynucleotide sequence is compared with the seed comprising the polynucleotide selected from the group depicted as SEQ ID NOS: 1; 2; 3; 4; 5 and 6, the seed are from plants which are grown subject to the same environmental growing conditions.

The present invention still further provides a polynucleotide sequence which is the complement of one which hybridises to a polynucleotide which consists of a sequence selected from the group depicted as SEQ ID NOS: 7; 8; and 9 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still comprises a region encoding a carotene desaturase and a further region encoding a phytoene synthase and when said polynucleotide sequence is inserted into plant material the seed of a plant regenerated from said material produce carotenoids amounting to at least 80% of the carotenoid content of a seed which comprises a polynucleotide selected from the group depicted as SEQ ID NOS: 7; 8 and 9. It is preferred that when the carotenoid content of the seed comprising said polynucleotide sequence is compared with the seed comprising the polynucleotide selected from the group depicted as SEQ ID NOS: 7; 8 and 9, the seed are from plants of substantially the same species. It is further preferred that when the carotenoid content of the seed comprising said polynucleotide sequence is compared with the seed comprising the polynucleotide selected from the group depicted as SEQ ID NOS: 7; 8 and 9, the seed are from plants which are substantially genetically identical save for the presence of said polynucleotide or said polynucleotide sequence.

In a particular embodiment the polynucleotide sequences according to the invention which are identified based on their hybridisation (under the conditions provided) to the sequences described in the Sequence Listing, encode the same proteins as those provided by the sequences in the Sequence Listing. In a further embodiment, said polynucleotide sequences may encode proteins which have the same or a similar function as the proteins encoded by the sequences in the Sequence Listing. In a still further embodiment, the proteins encoded by the polynucleotide sequence according to the invention comprise amino acid substitutions and/or deletions when compared to the porteins encoded by the sequences in the Sequence Listing. In a still further embodiment, said amino acid substitutions are “conservative” substitutions. A “conservative” substitution is understood to mean that the amino acid is replaced with an amino acid with broadly similar chemical properties. In particular “conservative” substitutions may be made between amino acids within the following groups: (i) Alanine and Glycine; (ii) Threonine and Serine; (ii) Glutamic acid and Aspartic acid; (iii) Arginine and Lysine; (iv) Asparagine and Glutamine; (v) Isoleucine and Leucine; (vi) Valine and Methionine; and (vii) Phenylalanine and Tryptophan.

The present invention still further provides a polynucleotide or a polynucleotide sequence as described above which when inserted into plant material, the seed of a plant regenerated from said material produces carotenoids at levels which are higher than those present in native like-seeds. The present invention still further provides a polynucleotide or a polynucleotide sequence as described above which when inserted into plant material, the seed of a plant regenerated from said material produces carotenoids at levels which are higher than those present in untransformed like-seeds.

In a particular embodiment, the carotenoids which are increased are selected from the group consisting of: lycopene; alpha-carotene; lutein; beta-carotene; zeaxanthin; beta-cryptoxanthin; antheraxanthin; violaxanthin; and neoxanthin or a combination thereof. In a further embodiment, the carotenoids which are increased are selected from the group consisting of: lycopene; alpha-carotene; lutein; beta-carotene; zeaxanthin; beta-cryptoxanthin; or a combination thereof. In a still further embodiment, the carotenoids which are increased are selected from the group consisting of: alpha-carotene; lutein; beta-carotene; zeaxanthin; beta-cryptoxanthin; or a combination thereof. In a still further embodiment, the carotenoids which are increased include at least phytoene and beta-carotene. In a still further embodiment, the carotenoids which are increased include at least beta-carotene. In a still further embodiment, the carotenoid which is increased is beta-carotene. In a still further embodiment the carotenoids which are increased are coloured carotenoids.

The present invention still further provides a polynucleotide or a polynucleotide sequence as described above wherein said seed is a rice seed. In a particular embodiment of the invention, before the seeds are analysed for their carotenoid content, the seeds are prepared prior to the analysis. Such preparation may include, for example, with respect to rice seed, “dehusking” and “polishing”. Furthermore, such preparation may involve the removal of those plant parts associated with the seed which plant parts are not normally intended for human consumption.

The amount of carotenoids in the seeds can be determined using techniques which are well known and available to the person skilled in the art. Such techniques include but are not necessarily limited to High Performance Liquid Chromatography (HPLC) analysis and spectrophotometry.

The present invention still further provides a polynucleotide or polynucleotide sequence as described above which further comprises a region which encodes a selectable marker. In a particular embodiment said selectable marker comprises a mannose-6-phosphate isomerase gene. In a further particular embodiment the selectable marker used is the mannose-6-phosphate isomerase gene according to the Positech™ selection system. In a specific embodiment said selectable marker is the one as described in European Patent/Application publication Number EP 0 896 063 and EP 0 601 092. Alternatively, the selectable marker used may, in particular, confer resistance to kanamycin, hygromycin or gentamycin. Further suitable selectable markers include genes that confer resistance to herbicides such as glyphosate-based herbicides (e.g. EPSPS genes such as in U.S. Pat. No. 5,510,471 or WO 00/66748) or resistance to toxins such as eutypine. Other forms of selection are also available such as hormone based selection systems such as the Multi Auto Transformation (MAT) system of Hiroyrasu Ebinuma et al. 1997. PNAS Vol. 94 pp 2117-2121; visual selection systems which use fluorescent proteins, β glucoronidase and any other selection system such as xylose isomerase and 2-deoxyglucose (2-DOG).

The present invention still further provides a polynucleotide or a polynucleotide sequence according to the invention which is codon optimised for expression in a particular plant species. In a particular embodiment the polynucleotide or polynucleotide sequence is codon optimised for expression in rice (Orzya sp.) or maize (Zea sp.). Such codon optimisation is well known to the person skilled in the art and the table below provides an example of the plant-preferred codons for rice and maize.

| Amino Acid | Rice preference | Maize preference |
|---|---|---|
| Alanine | GCC | GCC |
| Arginine | CGC | AGG |
| Asparagine | AAC | ACC |
| Aspartic Acid | GAC | GAC |
| Cysteine | TGC | TGC |
| Glutamine | CAG | CAG |
| Glutamic Acid | GAG | GAG |
| Glycine | GGC | GGC |
| Histidine | CAC | CAC |
| Isoleucine | ATC | ATC |
| Leucine | CTC | CTG |
| Lysine | AAG | AAG |
| Methionine | ATG | ATG |
| Phenylalanine | TTC | TTC |
| Proline | CCG | CCG |
| Serine | TCC | AGC |
| Threonine | ACC | ACC |
| Tryptophan | TGG | TGG |
| Tyrosine | TAC | TAC |
| Valine | GTG | GTG |

The present invention further provides a vector comprising a polynucleotide or a polynucleotide sequence as described above. In a particular embodiment of the invention said vector comprises a polynucleotide selected from the group depicted as SEQ ID NO: 1; 2; 3; 4; 5 and 6. In a particular embodiment of the invention said vector comprises a polynucleotide selected from the group depicted as SEQ ID NOS: 7; 8 and 9. In a particular embodiment said vector allows for replication of said polynucleotide or polynucleotide sequence in a bacterium.

The present invention still further provides a vector as described above which is a plant expression vector.

In a particular embodiment of the invention the sequence around the translational start position(s) of the phytoene synthase and/or said carotene desaturase encoding sequences as described above may be modified such that it is “Kozak” preferred. What is meant by this is well known to the skilled artisan. Examples of Kozak consensus sequences which are well known to the person skilled in the art include cagcc(atg) or agcc(atg). The phytoene synthase and/or said carotene desaturase encoding sequences as described above may also further comprise a sequence which provides for retention in a particular intracellular organelle.

In a further aspect of the present invention there is provided a method for increasing the carotenoid content of seeds comprising inserting into plant material a polynucleotide or a polynucleotide sequence or a vector as described above; and regenerating a seed-containing plant from said material and identifying the seeds which contain carotenoids at levels greater that those of a control like-seed.

The present invention still further provides a method for increasing the carotenoid content of seeds comprising inserting into plant material a polynucleotide comprising a sequence selected from the group depicted as SEQ ID NO: 1; 2; 3; 4; 5 and 6 and regenerating a seed containing plant from said material and identifying the seeds which contain carotenoids at levels greater that those of control like-seeds. In a particular embodiment of the invention, the seeds obtained by said method contain at least a sixty fold increase in carotenoids when compared to a control like-seed. In a further embodiment the seeds obtained by said method contain at least a one hundred fold increase in carotenoids when compared to control like-seeds. In a further embodiment the seeds obtained by said method contain at least a one hundred and fifty fold increase in carotenoids when compared to control like-seeds. In a further embodiment the seeds obtained by said method contain at least a two hundred fold increase in carotenoids when compared to control like-seeds. In a further embodiment the seeds obtained by said method contain at least a two hundred and fifty fold increase in carotenoids when compared to control like-seeds. In a further embodiment the seeds obtained by said method contain at least a three hundred fold increase in carotenoids when compared to control like-seeds. In a further embodiment the seeds obtained by said method contain at least a three hundred and fifty fold increase in carotenoids when compared to control like-seeds. In a further embodiment the seeds obtained by said method contain at least a four hundred fold increase in carotenoids when compared to control like-seeds. In a further embodiment the seeds obtained by said method contain at least a five hundred fold increase in carotenoids when compared to control like-seeds.

The present invention still further provides a method as described above wherein said seed contains carotenoids at a level of at least 3 μg/g of endosperm of said seed. In a particular embodiment said seed contains carotenoids at a level of at least 4 μg/g of endosperm of said seed. In a particular embodiment said seed contains carotenoids at a level of at least 5 μg/g of endosperm of said seed. In a particular embodiment said seed contains carotenoids at a level of at least 6 μg/g of endosperm of said seed. In a particular embodiment said seed contains carotenoids at a level of at least 7 μg/g of endosperm of said seed. In a particular embodiment said seed contains carotenoids at a level of at least 8 μg/g of endosperm of said seed. In a particular embodiment said seed contains carotenoids at a level of at least 9 μg/g of endosperm of said seed. In a particular embodiment said seed contains carotenoids at a level of at least 10 μg/g of endosperm of said seed. In a particular embodiment said seed contains carotenoids at a level of at least 15 μg/g of endosperm of said seed. In a particular embodiment said seed contains carotenoids at a level of at least 20 μg/g of endosperm of said seed. In a particular embodiment said seed contains carotenoids at a level of at least 25 μg/g of endosperm of said seed. In a particular embodiment said seed contains carotenoids at a level of at least 30 μg/g of endosperm of said seed. In a particular embodiment said seed contains carotenoids at a level of at least 35 μg/g of endosperm of said seed. In a particular embodiment said seed contains carotenoids at a level of at least 40 μg/g of endosperm of said seed. In a particular embodiment said seed contains carotenoids at a level of at least 45 μg/g of endosperm of said seed. In a particular embodiment said seed contains carotenoids at a level of at least 50 μg/g of endosperm of said seed. In a particular embodiment said seed contains carotenoids at a level of at least 55 μg/g of endosperm of said seed. In a particular embodiment said seed contains carotenoids at a level of at least 60 μg/g of endosperm of said seed. In a particular embodiment said seed contains carotenoids at a level of at least 65 μg/g of endosperm of said seed.

The present invention still further provides a method for increasing the carotenoid content of seeds comprising inserting into plant material a polynucleotide sequence as described above and regenerating a seed-containing plant from said material and identifying the seed which contains carotenoids amounting to at least 80% of the carotenoid content of a seed which comprises a polynucleotide selected from the group depicted as SEQ ID NOS: 1; 2; 3; 4; 5 and 6. In a further embodiment, the seed of a plant regenerated from said material produce carotenoids amounting to at least 85% of the carotenoid content of a seed which comprises a polynucleotide selected from the group depicted as SEQ ID NOS: 1; 2; 3; 4; 5 and 6. In a still further embodiment, the seed of a plant regenerated from said material produce carotenoids amounting to at least 90% of the carotenoid content of a seed which comprises a polynucleotide selected from the group depicted as SEQ ID NOS: 1; 2; 3; 4; 5 and 6. In a still further embodiment, the seed of a plant regenerated from said material produce carotenoids amounting to at least 95% of the carotenoid content of a seed which comprises a polynucleotide selected from the group depicted as SEQ ID NOS: 1; 2; 3; 4; 5 and 6. In a still further embodiment, the seed of a plant regenerated from said material produce carotenoids amounting to at least 100% of the carotenoid content of a seed which comprises a polynucleotide selected from the group depicted as SEQ ID NOS: 1; 2; 3; 4; 5 and 6. In a particular embodiment the polynucleotide sequence provides for a percentage of the carotenoid content of seed as described above wherein the seed with which the comparison is made comprises the polynucleotide depicted as SEQ ID NO: 1. In a particular embodiment the polynucleotide sequence provides for a percentage of the carotenoid content of seed as described above wherein the seed with which the comparison is made comprises the polynucleotide depicted as SEQ ID NO: 2. In a particular embodiment the polynucleotide sequence provides for a percentage of the carotenoid content of seed as described above wherein the seed with which the comparison is made comprises the polynucleotide depicted as SEQ ID NO: 3. In a particular embodiment the polynucleotide sequence provides for a percentage of the carotenoid content of seed as described above wherein the seed with which the comparison is made comprises the polynucleotide depicted as SEQ ID NO: 4. In a particular embodiment the polynucleotide sequence provides for a percentage of the carotenoid content of seed as described above wherein the seed with which the comparison is made comprises the polynucleotide depicted as SEQ ID NO: 6.

The present invention still further provides a method for increasing the carotenoid content of seeds comprising inserting into plant material a polynucleotide comprising a sequence selected from the group depicted as SEQ ID NOS: 7; 8 and 9 and regenerating a seed containing plant from said material and identifying the seeds which contain carotenoids at levels greater that those of control like-seeds. In a particular embodiment of the invention, the seeds obtained by said method contain at least a fifty fold increase in carotenoids when compared to a control like-seed.

The present invention still further provides a method as described above wherein said seed contains carotenoids at a level of at least 3 μg/g of endosperm of said seed.

The present invention still further provides a method for increasing the carotenoid content of seeds comprising inserting into plant material a polynucleotide sequence as described above and regenerating a seed-containing plant from said material and identifying the seed which contains carotenoids amounting to at least 80% of the carotenoid content of a seed which comprises a polynucleotide selected from the group depicted as SEQ ID NOS: 7; 8 and 9.

The present invention still further provides a method as described above wherein the carotenoids which are increased are selected from the group consisting of: lycopene; alpha-carotene; lutein; beta-carotene; zeaxanthin; beta-cryptoxanthin; antheraxanthin; violaxanthin; and neoxanthin or a combination thereof. In a further embodiment, the carotenoids which are increased are selected from the group consisting of: lycopene; alpha-carotene; lutein; beta-carotene; zeaxanthin; beta-cryptoxanthin; or a combination thereof. In a still further embodiment, the carotenoids which are increased are selected from the group consisting of: alpha-carotene; lutein; beta-carotene; zeaxanthin; beta-cryptoxanthin; or a combination thereof. In a still further embodiment, the carotenoids which are increased include at least phytoene and beta-carotene. In a still further embodiment, the carotenoids which are increased include at least beta-carotene. In a still further embodiment, the carotenoid which is increased is beta-carotene. In a still further embodiment the carotenoids which are increased are coloured carotenoids.

The polynucleotide or polynucleotide sequence or vector as described above may be inserted into plant material by plant transformation techniques that are well known to the person skilled in the art. Such techniques include but are not limited to particle mediated biolistic transformation, Agrobacterium-mediated transformation, protoplast transformation (optionally in the presence of polyethylene glycols); sonication of plant tissues, cells or protoplasts in a medium comprising the polynucleotide or vector; micro-insertion of the polynucleotide or vector into totipotent plant material (optionally employing the known silicon carbide “whiskers” technique), electroporation and the like. In a particular embodiment of the invention rice plant material is transformed in accordance with the methods described in the Examples disclosed herein. In a particular embodiment the Agrobacterium that is used is a strain that has been modified to reduce the possibility of recombination between sequences having a high degree of similarity within the TDNA region of the Agrobacterium. Furthermore, techniques and elements such as those referred to in WO99/01563, U.S. Pat. No. 6,265,638, U.S. Pat. No. 5,731,179 and U.S. Pat. No. 5,591,616 may be employed as part of the transformation process.

The present invention still further provides seed obtained or obtainable by a method as described above. In a specific embodiment said seed are rice seed. In a further embodiment said seeds are maize seeds.

Throughout this specification the terms “seed” and “seeds” may be interchanged with the terms “grain” or “grains”. In particular, the terms “seed” and “seeds” refers to edible seeds or seed parts, in particular, seed endosperm.

The present invention still further comprises a plant which comprises a seed according to the preceding paragraph.

The present invention further provides a plant or plant material which comprises a polynucleotide or a polynucleotide sequence or a vector as described above. In a particular embodiment said plant or plant material is a rice plant or rice plant material or a maize plant or maize plant material. In a still further embodiment the plant or plant material of the present invention is selected from the group consisting watermelon, melon, mango, soybean, cotton, tobacco, sugar beet, oilseed rape, canola, flax, sunflower, potato, tomato, alfalfa, lettuce, maize, wheat, sorghum, rye, bananas, barley, oat, turf grass, forage grass, sugar cane, pepper, pea, field bean, rice, pine, poplar, apple, peach, grape, strawberry, carrot, cabbage, onion, citrus, cereal or nut plants or any other horticultural crops. In a specific embodiment said plant is a rice plant.

The present invention still further provides a plant or seed according to the invention which further comprises a gene which gene encodes an enzyme which is capable of converting carotene to a retinoid. An example of such a gene is the gene encoding β-carotene dioxygenase as described in WO01/48162 and/or WO01/48163.

The present invention still further provides a plant according to the invention which further comprises a polynucleotide which provides for a trait selected from the group consisting of: insect resistance and/or tolerance; nematode resistance and/or tolerance; herbicide resistance and/or tolerance; improved resistance and/or tolerance to stress; a substance having pharmaceutical activity and/or any other desired agronomic trait.

The present invention still further provides a plant according to the invention which further comprises a polynucleotide which provides for a further enhancement of isoprenoid biosynthesis and/or carotenoid accumulation in the plant. In a particular embodiment said polynucleotide provides for a transcription factor which provides for a further enhancement of isoprenoid biosynthesis and/or carotenoid accumulation in the plant.

The present invention still further provides a plant according to the invention which further comprises a polynucleotide which provides for an increase in plastids within the plant. In a particular embodiment said polynucleotide comprises the PhyA gene from oats or arabidopsis which genes are well known to the person skilled in the art. In a further embodiment said polynucleotide comprises the Hp1 or Hp2 gene from tomato which are also well known the person skilled in the art.

The present invention still further provides a molecular marker which marker is capable of identifying plant material which comprises a sequence selected from the group depicted as SEQ ID NOS: 1 to 9 wherein said molecular marker comprises at least about 25 contiguous nucleotides of a sequence selected from the group consisting of SEQ ID NOS: 1 to 9.

The present invention still further provides a method for identifying plant material according to the invention via the use, for example, of the polymerase chain reaction (PCR). Suitable primers may be designed using parameters well known to those skilled in the art and based on the sequences listed in the Sequence Listing. The person skilled in the art is well versed in nucleic acid extraction techniques, and once a test sample has been isolated, it can be analysed for the presence of the sequence according to the invention using techniques that are well known in the art. These include, but are not limited to, PCR, RAPIDS, RFLPs and AFLPs.

The present invention still further provides a kit which kit comprises a means for obtaining a test sample and a means for detecting the presence of the sequences of the invention within said test sample. Kits may also be generated that are suitable for testing the carotenoid content of a test sample and this may optionally be combined with the features of a kit as described in the preceding sentence.

In a further aspect of the present invention there is provided the use of a polynucleotide, polynucleotide sequence or a vector as described above in a method for the production of seeds containing increased carotenoids. In a particular embodiment the present invention provides the use of a polynucleotide selected from the group depicted as SEQ ID NOS: 1; 2; 3; 4; 5 and 6 for the production of seeds which contain carotenoids at levels greater that those of a control like-seed.

In a further aspect of the present invention there is provided the use of a polynucleotide, polynucleotide sequence or a vector as described above in a method for the production of seeds containing increased carotenoids. In a particular embodiment the present invention provides the use of a polynucleotide selected from the group depicted as SEQ ID NOS: 7; 8 and 9 for the production of seeds which contain carotenoids at levels greater that those of a control like-seed.

In a further aspect of the invention there is provided the use of a polynucleotide, polynucleotide sequence or a vector as described above in a method of producing a plant which comprises said polynucleotide, said polynucleotide sequence or said vector.

In a further aspect of the invention there is provided the use of a polynucleotide selected from the group depicted as SEQ ID NOS: 1; 2; 3; 4; 5 and 6 in a method for the production of a plant comprising said polynucleotide.

In a further aspect of the invention there is provided the use of a polynucleotide selected from the group depicted as SEQ ID NOS: 7; 8 and 9 in a method for the production of a plant comprising said polynucleotide.

In a further aspect of the invention there is provided a method for increasing the carotenoid content of seeds comprising inserting into plant material (a) a first polynucleotide which comprises as operably linked components (i) a promoter which provides for seed preferred expression; and (ii) a nucleotide sequence derived from a bacterium which sequence encodes a carotene desaturase; and (iii) a transcription termination region; and (b) a second polynucleotide which comprises as operably linked components (i) a promoter which provides for seed preferred expression; and (ii) a nucleotide sequence encoding a phytoene synthase which sequence is derived from maize (Zea sp.) or rice (Orzya sp.); and (iii) a transcription termination region; and (c) regenerating a seed containing plant from said material and identifying the seeds which contain carotenoids at levels greater that those of control like-seeds.

In a further aspect of the invention there is provided a method for increasing the carotenoid content of seeds comprising inserting into plant material (a) a first polynucleotide which comprises as operably linked components (i) a promoter which provides for seed preferred expression; and (ii) a nucleotide sequence derived from a bacterium which sequence encodes a carotene desaturase; and (iii) a transcription termination region; and (b) a second polynucleotide which comprises as operably linked components (i) a promoter which provides for seed preferred expression; and (ii) a nucleotide sequence encoding a phytoene synthase which sequence is derived from tomato (Lycopersicon sp.) or pepper (Capsicum sp.); or a bacterium; and (iii) a transcription termination region; and (c) regenerating a seed containing plant from said material and identifying the seeds which contain carotenoids at levels greater that those of control like-seeds.

In a particular embodiment, step (a) of the preceding paragraph is performed prior to step (b). In a further embodiment, step (b) of the preceding paragraph is performed prior to step (a). In a still further embodiment, the promoter, the sequence encoding said carotene desaturase, the sequence encoding phytoene synthase and the terminator region are derived from the sequences depicted in the Sequence Listing. In a still further embodiment said carotene desaturase is the CrtI gene from Erwinia sp. In a still further embodiment the carotene desaturase comprises or consists of the sequence depicted as SEQ ID NO: 18 or 19. In a further embodiment said phytoene synthase is derived from maize or rice. In a still further embodiment said phytoene synthase is from maize or rice. In a still further embodiment said phytoene synthase comprises or consists of a sequence selected from the group depicted as SEQ ID NOS: 10; 11; 12; and 13.

In a further aspect of the invention there is provided a method for increasing the carotenoid content of seeds comprising crossing (a) a first plant comprising a polynucleotide which comprises as operably linked components (i) a promoter which provides for seed preferred expression; and (ii) a nucleotide sequence derived from a bacterium which sequence encodes a carotene desaturase; and (iii) a transcription termination region; with (b) a further plant comprising a polynucleotide which comprises as operably linked components (i) a promoter which provides for seed preferred expression; and (ii) a nucleotide sequence encoding a phytoene synthase which sequence is derived from maize (Zea sp.) or rice (Orzya sp.); and (iii) a transcription termination region; and (c) harvesting seed from the female parent of the thus crossed plants; and (d) growing said seed to produce plants comprising further seeds and identifying said further seeds which contain carotenoids at levels greater that those of control like-seeds.

In a further aspect of the invention there is provided a method for increasing the carotenoid content of seeds comprising crossing (a) a first plant comprising a polynucleotide which comprises as operably linked components (i) a promoter which provides for seed preferred expression; and (ii) a nucleotide sequence derived from a bacterium which sequence encodes a carotene desaturase; and (iii) a transcription termination region; with (b) a further plant comprising a polynucleotide which comprises as operably linked components (i) a promoter which provides for seed preferred expression; and (ii) a nucleotide sequence encoding a phytoene synthase which sequence is derived from tomato (Lycopersicon sp.) or pepper (Capsicum sp.); or a bacterium; and (iii) a transcription termination region; and (c) harvesting seed from the female parent of the thus crossed plants; and (d) growing said seed to produce plants comprising further seeds and identifying said further seeds which contain carotenoids at levels greater that those of control like-seeds.

In a still further embodiment, the promoter, the sequence encoding said carotene desaturase, the sequence encoding phytoene synthase and the terminator region are obtainable from the sequences depicted in the Sequence Listing. In a still further embodiment said carotene desaturase is the CrtI gene from Erwinia sp. In a still further embodiment the carotene desaturase comprises or consists of the sequence depicted as SEQ ID NO: 18 or 19. In a further embodiment said phytoene synthase is derived from maize or rice. In a still further embodiment said phytoene synthase is from maize or rice. In a still further embodiment said phytoene synthase comprises or consists of a sequence selected from the group depicted as SEQ ID NOS: 10; 11; 12 and 13.

In a further aspect of the invention there is provided a polynucleotide which comprises (a) a region which comprises as operably linked components (i) a promoter which provides for seed preferred expression; and (ii) a nucleotide sequence derived from a bacterium which sequence encodes a carotene desaturase or a nucleotide sequence which encodes a carotene desaturase derived from a plant selected from the group consisting of: tomato (Lycopersicon sp.); pepper (Capsicum sp.); maize (Zea sp.); rice (Orzya sp.); and (iii) a transcription termination region; and (b) a further region which comprises as operably linked components (i) a promoter which provides for seed preferred expression; and (ii) a nucleotide sequence encoding a phytoene synthase which sequence is derived from a bacterium, or from a plant selected from the group consisting of: tomato (Lycopersicon sp.); pepper (Capsicum sp.); maize (Zea sp.); rice (Orzya sp.); and (iii) a transcription termination region; and (c) a still further region which comprises as operably linked components (i) a promoter which provides for seed preferred expression; and (ii) a nucleotide sequence encoding a zeta-carotene desaturase (ZDS) derived from a bacterium, or from a plant selected from the group consisting of: tomato (Lycopersicon sp.); pepper (Capsicum sp.); maize (Zea sp.); rice (Orzya sp.); and (iii) a transcription termination region. In a particular embodiment the carotene desaturase and phytoene synthase is derived from maize (Zea sp.) and zeta-carotene desaturase (ZDS) is derived from pepper (Capsicum sp.). In a further embodiment the carotene desaturase and phytoene synthase is from maize (Zea sp.) and the zeta-carotene desaturase (ZDS) is from pepper (Capsicum sp.).

The polynucleotides as described above may be used to identify polynucleotide sequences providing for a like-function, based on the hybrisation conditions described above. These polynucleotides and polynucleotide sequences which comprise the zeta-carotene desaturase may also be used in methods for increasing the carotenoid content of seeds in a manner analogous to the methods described above.

In a further aspect of the invention there is provided a polynucleotide which comprises as operably linked components (i) a promoter which provides for seed preferred expression; and (ii) a nucleotide sequence derived from a bacterium which sequence encodes a carotene desaturase or a nucleotide sequence which encodes a carotene desaturase derived from a plant selected from the group consisting of: tomato (Lycopersicon sp.); pepper (Capsicum sp.); maize (Zea sp.); rice (Orzya sp.); and (iii) a nucleotide sequence encoding a phytoene synthase which sequence is derived from a plant selected from the group consisting of: tomato (Lycopersicon sp.); pepper (Capsicum sp.); maize (Zea sp.); rice (Orzya sp.) or a bacterium; and (iv) a nucleotide sequence encoding a zeta-carotene desaturase (ZDS) derived from a bacterium or from a plant selected from the group consisting of: tomato (Lycopersicon sp.); pepper (Capsicum sp.); maize (Zea sp.); rice (Orzya sp.); and (v) a transcription termination region.

Any of the regions described in this specification may be separated by a region which provides for a self-processing polypeptide which is capable of separating the proteins such as the self-processing polypeptide described in U.S. Pat. No. 5,846,767 or any similarly functioning element. Alternatively the regions may be separated by a sequence which acts as a target site for an external element which is capable of separating the protein sequences. Alternatively the polynucleotide may provide for a polyprotein which comprises a plurality of protein functions. In a further embodiment of the present invention the proteins of the polyprotein may be arranged in tandem. The person skilled in the art will appreciate that when a polynucleotide is generated which encodes such a polyprotein, expression of such a polyprotein may be achieved via the use of a single promoter which promoter is described herein.

All of the polynucleotides and polynucleotide sequences described throughout this specification can be isolated and constructed using techniques that are well known to the person skilled in the art. For example, the polynucleotides can be synthesised using standard polynucleotide synthesisers. Such synthetic polynucleotides can be synthesised and then ligated to form the longer polynucleotides according to the invention. The polynucleotides and polynucleotide sequences can also be isolated from other constructs/vectors which contain said sequences and then be inserted into further constructs/vectors to produce the ones according to the invention. The sequences can also be isolated from libraries, for example cDNA and gDNA, using the sequence information provided in the Sequence Listing for the creation of suitable probes/primers for the purpose of identifying said sequences from said libraries. Once isolated, the sequences can be assembled to create the polynucleotides and polynucleotide sequences according to the invention. The sequences according to the invention can also be used to identify like-sequences in accordance with the hybridisation conditions described above. In identifying such like-sequences the person skilled in the art may wish to identify like-sequences of one or more of the component parts of the sequences depicted in the Sequence Listing and then subsequently assemble the like-sequences in a manner similar to the arrangement of the sequences depicted in the Sequence Listing. For example, it may be desired to modify the region encoding phytoene synthase gene only. Once the modified phytoene synthase encoding sequence had been identified, it could be used to replace the phytoene synthase encoding sequence in one of the sequences depicted in the Sequence Listing. Alternatively, the modified phytoene synthase may be used in the creation of a new sequence wherein all the component parts are the same as a sequence in the Sequence Listing save for the phytoene synthase. In a further example, all of the components may be modified and then each of the modified components is arranged in the same manner as the sequences of the Sequence Listing, for example, promoter-intron-target sequence-carotene desaturase-terminator-promoter-intron-(target sequence)-phytoene synthase-terminator. The degree of modification will affect the ability of the thus modified sequence to hybridise to a sequence in the Sequence Listing under the conditions described above.

The present invention further provides a plant which comprises a polynucleotide or polynucleotide sequence as described above. In a particular embodiment said plant is a rice or a maize plant.

In a further aspect of the present invention there is provided a polynucleotide or a polynucleotide sequence as described above wherein the promoter(s) are tissue preferred and/or organ preferred. In a particular embodiment the promoter(s) provide for prefferential expression in fruit. In a further embodiment said promoter(s) provide for high expression in fruit. In a still further embodiment said fruit is a banana fruit. In a further aspect of the present invention there is provided a polynucleotide comprising: (a) a region which comprises as operably linked components (i) a promoter which provides for fruit preferred expression; and (ii) a nucleotide sequence derived from a bacterium which sequence encodes a carotene desaturase; and (iii) a transcription termination region; and (b) a further region which comprises as operably linked components (i) a promoter which provides for fruit preferred expression; and (ii) a nucleotide sequence encoding a phytoene synthase which sequence is derived from maize (Zea sp.) or rice (Orzya sp.); and (iii) a transcription termination region. In a still further embodiment there is provided a method for increasing the carotenoid content of fruit comprising inserting into plant material a polynucleotide comprising: (a) a region which comprises as operably linked components (i) a promoter which provides for fruit preferred expression; and (ii) a nucleotide sequence derived from a bacterium which sequence encodes a carotene desaturase; and (iii) a transcription termination region; and (b) a further region which comprises as operably linked components (i) a promoter which provides for fruit preferred expression; and (ii) a nucleotide sequence encoding a phytoene synthase which sequence is derived from maize (Zea sp.) or rice (Orzya sp.); and (iii) a transcription termination region and regenerating a fruit-containing plant from said material and identifying the fruit which contain carotenoids at levels greater than those of control like-fruit. The present invention still further provides polynucleotides which comprise the phytoene synthase encoding sequences and carotene desaturase encoding sequences mentioned above which sequences are operably linked to promoters which provide for fruit preferred or tissue or organ preferred expression. Such suitable promoters may be identified by the person skilled in the art.

In a further aspect of the present invention there is provided the use of a polynucleotide or a polynucleotide sequence as described above in a method for the production of plants which are resistant and/or tolerant to a herbicide.

In a still further aspect of the present invention there is provided a method for the production of a plant that is resistant and/or tolerant to a herbicide comprising inserting into plant material a polynucleotide or a polynucleotide sequence as described above and regenerating a morphologically normal plant from said material. The herbicide resistance and/or tolerance of the plant containing the polynucleotide or polynucleotide sequence of the invention can be compared to a control like-plant. The term control like-plant relates to plants which are substantially similar to those according to the invention but which control like-plant does not contain the polynucleotides or polynucleotide sequences according to the invention. Typically, a control like-plant will comprise a plant of the same or similar plant species which control like-plant is a native plant or which has not been transformed.

In a further aspect of the present invention there is provided a polynucleotide comprising the sequence depicted as SEQ ID NO: 13. In a particular embodiment there is provided a polynucelotide which consists of the sequence depicted as SEQ ID NO: 13. The present invention still further provides a polynucleotide which encodes the protein depicted as SEQ ID NO: 14. The present invention still further provides a polynucleotide sequence which has at least 87% identity to the sequence depicted as SEQ ID NO: 13 wherein said sequence still encodes a phytoene synthase. The present invention still further provides a polynucleotide sequence which has at least 90% identity to the sequence depicted as SEQ ID NO: 13 wherein said sequence still encodes a phytoene synthase. The present invention still further provides a polynucleotide sequence which has at least 91% identity to the sequence depicted as SEQ ID NO: 13 wherein said sequence still encodes a phytoene synthase. The present invention still further provides a polynucleotide sequence which has at least 92% identity to the sequence depicted as SEQ ID NO: 13 wherein said sequence still encodes a phytoene synthase. The present invention still further provides a polynucleotide sequence which has at least 93% identity to the sequence depicted as SEQ ID NO: 13 wherein said sequence still encodes a phytoene synthase. The present invention still further provides a polynucleotide sequence which has at least 94% identity to the sequence depicted as SEQ ID NO: 13 wherein said sequence still encodes a phytoene synthase. The present invention still further provides a polynucleotide sequence which has at least 95% identity to the sequence depicted as SEQ ID NO: 13 wherein said sequence still encodes a phytoene synthase. The present invention still further provides a polynucleotide sequence which has at least 96% identity to the sequence depicted as SEQ ID NO: 13 wherein said sequence still encodes a phytoene synthase. The present invention still further provides a polynucleotide sequence which has at least 97% identity to the sequence depicted as SEQ ID NO: 13 wherein said sequence still encodes a phytoene synthase. The present invention still further provides a polynucleotide sequence which has at least 98% identity to the sequence depicted as SEQ ID NO: 13 wherein said sequence still encodes a phytoene synthase. The present invention still further provides a polynucleotide sequence which has at least 99% identity to the sequence depicted as SEQ ID NO: 13 wherein said sequence still encodes a phytoene synthase.

The present invention still further provides a protein having the sequence depicted as SEQ ID NO: 14 or a variant which has at least 82% identity to SEQ ID NO: 14 wherein said variant still provides for phytoene synthase activity. In a further embodiment said variant has at least 85% identity to SEQ ID NO: 14 wherein said variant still provides for phytoene synthase activity. In a still further embodiment said variant has at least 90% identity to SEQ ID NO: 14 wherein said variant still provides for phytoene synthase activity. In a still further embodiment said variant has at least 91% identity to SEQ ID NO: 14 wherein said variant still provides for phytoene synthase activity. In a still further embodiment said variant has at least 92% identity to SEQ ID NO: 14 wherein said variant still provides for phytoene synthase activity. In a still further embodiment said variant has at least 93% identity to SEQ ID NO: 14 wherein said variant still provides for phytoene synthase activity. In a still further embodiment said variant has at least 94% identity to SEQ ID NO: 14 wherein said variant still provides for phytoene synthase activity. In a still further embodiment said variant has at least 95% identity to SEQ ID NO: 14 wherein said variant still provides for phytoene synthase activity. In a still further embodiment said variant has at least 96% identity to SEQ ID NO: 14 wherein said variant still provides for phytoene synthase activity. In a still further embodiment said variant has at least 97% identity to SEQ ID NO: 14 wherein said variant still provides for phytoene synthase activity. In a still further embodiment said variant has at least 98% identity to SEQ ID NO: 14 wherein said variant still provides for phytoene synthase activity. In a still further embodiment said variant has at least 99% identity to SEQ ID NO: 14 wherein said variant still provides for phytoene synthase activity. In a still further embodiment there is provided a polynucleotide which encodes said variant. By phytoene synthase activity it is meant that the variant protein has the same or a similar function to the protein depicted as SEQ ID NO: 14. The percentage of sequence identity for proteins is determined by comparing two optimally aligned sequences over a comparison window, wherein the portion of the amino acid sequence in the comparison window may comprise additions or deletions (i.e. gaps) as compared to the initial reference sequence (which does not comprise additions or deletions) for optimal alignment of the two sequences. The percentage is calculated by determining the number of positions at which the identical amino acid residue occurs in both sequences to yield the number of match positions, dividing the number of match positions by the total number of positions in the window of comparison and multiplying the result by 100 to yield the percentage of sequence identity. Optimal alignment of sequences for comparison may also be conducted by computerised implementations of known algorithms such as Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. There are also algorithms available to the person skilled in the art that enable a calculation of the percentage sequence identity between polynucleotide sequences. The variant may differ from the protein depicted as SEQ ID NO: 14 in particular by conservative substitutions. Such conservative substitutions are described above.

The present invention will now be described by way of the following non-limiting examples with reference to the following Figures and Sequence Listing of which:

SEQ ID NO: 1=12423=Glu-Cat-SSU-crtI-Nos-Glu-Cat-Psy (Maize-gb)-nos

SEQ ID NO: 2=12421=Glu-Cat-SSU-crtI-Nos-Glu-Cat-Psy (Maize-E1B)-nos

SEQ ID NO: 3=12422=Glu-SSU-crtI-Nos-Glu-Psy (Maize-E1B)-nos

SEQ ID NO: 4=12424=Glu-SSU-crtI-Nos-Glu-Psy (Maize-gb)-nos

SEQ ID NO: 5=Glu-Cat-SSU-crtI-Nos-Glu-Cat-Psy (Maize)-nos

SEQ ID NO: 6=11586=Glu-Cat-SSU-crtI-Nos-Glu-Cat-Psy (Rice)-nos

SEQ ID NO: 7=7651=Glu-Cat-SSU-crtI-Nos-Glu-Cat-Psy (Pepper)-nos

SEQ ID NO: 8=7650=Glu-Cat-SSU-crtI-Nos-Glu-Cat-Psy (Tomato)-nos

SEQ ID NO: 9=Glu-Cat-SSU-crtI-Nos-Glu-Cat-SSU-Psy (crtB)-nos

SEQ ID NO: 10=Phytoene synthase gb (Maize)

SEQ ID NO: 11=Phytoene synthase (Maize) from SEQ ID NO 5 above

SEQ ID NO: 12=Phytoene synthase E1B (Maize)

SEQ ID NO: 13=Phytoene synthase (Rice)

SEQ ID NO: 14=Phytoene synthase (Rice) PROTEIN

SEQ ID NO: 15=Phytoene synthase (Pepper)

SEQ ID NO: 16=Phytoene synthase (Tomato)

SEQ ID NO: 17=Phytoene synthase (Erwinia crtB)

SEQ ID NO: 18=Carotene desaturase (Erwinia crtI) used in SEQ ID NOS: 1-4

SEQ ID NO: 19=Carotene desaturase (Erwinia crtI)

SEQ ID NO: 20=Glutelin seed preferred promoter

SEQ ID NO: 21=Prolamin seed preferred promoter

SEQ ID NO: 22=Intron from catalase gene

SEQ ID NO: 23=Transit peptide (Small sub-unit Rubisco)

SEQ ID NO: 24=Transcription termination region from nopaline synthase gene

SEQ ID NO: 25=Transcription termination region from 35S CaMV

SEQ ID NO: 26=Transcription termination region from proteinase inhibitor form potato

SEQ ID NO: 27=Carotene desaturase (Tomato)

SEQ ID NO: 28=Carotene desaturase (Pepper)

SEQ ID NO: 29=Carotene desaturase (Maize)

SEQ ID NO: 30=Carotene desaturase (Rice)

SEQ ID NO: 31=Zeta-carotene desaturase (Tomato)

SEQ ID NO: 32=Zeta-carotene desaturase (Pepper)

SEQ ID NO: 33=Zeta-carotene desaturase (Maize)

SEQ ID NO: 34=Zeta-carotene desaturase (Rice)

SEQ ID NOS: 35 to 38=Primers

Glu=Glutelin seed preferred promoter

Pro=Prolamin seed preferred promoter

Cat=Intron from catalase gene

SSU=Transit peptide (Small sub-unit Rubisco)

CAI=Carotene desaturase from Erwinia

Pds=Carotene desaturase (source indicated in parenthesis)

Psy=Phytoene synthase (source indicated in parenthesis)

Zds=Zeta-carotene desaturase

Pds=Phytoene desaturase

Nos=Transcription termination region from nopaline synthase gene

35S term=Transcription termination region from 35S CaMV

PotP1-II term=Transcription termination region from proteinase inhibitor form potato

FIG. 1=Part of carotenoid biosynthesis pathway starting from GGPP (Geranylgeranyl diphosphate).

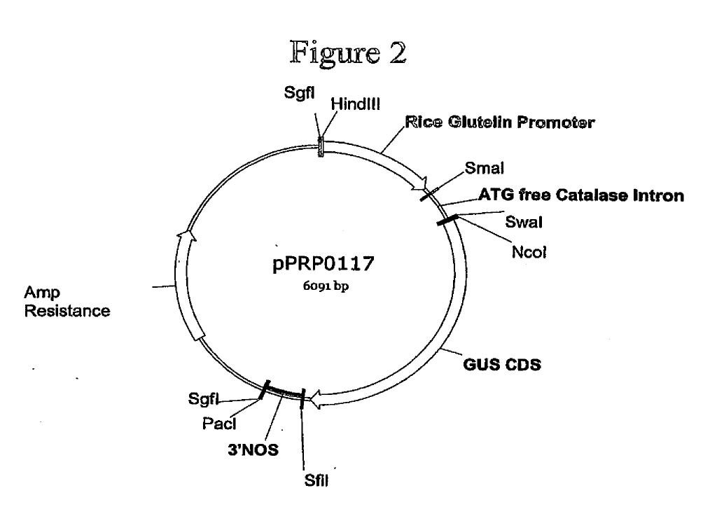

FIG. 2=Construct pPRP0117.

### EXAMPLES

General molecular biology methods are carried out according to Sambrook et al (1989) ‘Molecular cloning: A laboratory Manual, 2nd Edition. Cold Spring Harbour Lab. Press.

### 1.0 Construction of Plant Binary Vectors

References for the gene sequences below are given as they are listed on the EMBL database. This database is maintained and distributed by the European Bioinformatics Institute (Patricia Rodriguez-Tomé, Peter J. Stoehr, Graham N. Cameron and Tomas P. Flores, “The European Bioinformatics Institute (EBI) databases”, Nucleic Acids Res. 24:(6-13), 1996, www.ebi.ac.uk.)

A pUC based vector pPRP0117 (FIG. 2) was used for cloning of all the plant transformation vectors. This contains the nucleotides −806 to +33 of the rice glutelin gene as a promoter (Y00687), the first intron of the catalase-1 gene from castor bean altered to remove the ATG sequences, a gus coding region and a nos terminator. The gus coding sequence was removed by digestion of pPRP0117 with Nco1 and Sfi1 and replaced by the coding regions of the carotenoid phytoene synthase genes or phytoene desaturase genes, or removed as a gus::nos cassette by digestion of pPRP0117 with Nco1 and Pac1 and replaced by a carotenoid coding region/nos terminator fusion as described below.

1.1 Construction of the pPRP0117+CrtI Vector

A cassette of the signal peptide of the small subunit of pea ribulose bisphosphate carboxylase (SSU) (X00806) fused to the bacterial phytoene desaturase CrtI (D90087) and a nos terminator was cloned into the Nco1 and Pac1 sites of pPRP0117. The crtI sequence in constructs 7651, 7650 and 11586 had 9 nucleotides extra (3 alanines) inserted after the first ATG in order to incorporate a NotI restriction site for cloning purposes.

1.2 Construction of the pJH0104HygCrtI Binary Vector

The SgfI Gt::intron::SSUcrtI::nos cassette was cloned into the Pad site of the binary vector pJH0104+Hyg (which contains a hygromycin resistance gene for antibiotic selection) to give the construct pJH0104HygSSUCrtI.

1.3 Pepper psy+crtI Construct 7651

The catalase intron and pepper psy (X68017) were separately amplified by PCR using primers that overlap both sequences, and then fused by recombinant PCR, and cloned into the EcoRI and SfiI sites of pPRP0117. The Gt::intron::pepper psy::nos cassette was recovered with SgfI digestion and cloned into the Pad site of pJH0104HygCrtI to give the construct 7651.

1.4 Tomato psy+crtI Construct 7650

The catalase intron and tomato psy (Y00521) were separately amplified by PCR using primers that overlap both sequences, and then fused by recombinant PCR and cloned into the EcoRI and SfiI sites of pPRP0117. The Gt::intron::tomato psy::nos cassette was recovered by Sgf1 and cloned into the Pad site of pJH0104HygCrtI to give the construct 7650.

1.5 Rice psy+crtI Construct 11586

PolyA mRNA was extracted from rice leaves (Asanohikari). First strand synthesis of Rice psy cDNA was synthesized using the antisense primer 5′ cgtcggcctgcatggccctacttctggctatttctcagtg 3′ (SEQ ID NO: 35) and cDNA was then obtained by PCR amplification with this antisense primer and the sense primer 5′ctgtccatggcggccatcacgctcct 3′ (SEQ ID NO: 36). This was digested with NcoI and SfiI and cloned into pPRP0117. The Gt::intron::rice psy::nos fragment was transferred to the binary vector pJH0104Hyg. A HindIII/PacI Gt::intron::SSUcrtI::nos cassette was blunt ended and cloned into the Pme1 site of the pJH0104HygRicepsy vector to give the construct 11586.

1.6 Maize psy (E1B)+crtI Construct 12421

PolyA mRNA was extracted from maize leaves. First strand synthesis of maize psy (sequence designated “E1B”) cDNA was synthesized using the antisense primer 5′ cgatggcctgcatggccctaggtctggccatttctcaatg 3′ (SEQ ID NO: 37) and cDNA was then obtained by PCR amplification with this antisense primer and the sense primer 5′ taggataagatagcaaatccatggccatcata 3′ (SEQ ID NO: 38). This was digested with NcoI and SfiI and cloned into the Nco1 and Sfi1 sites of a pPRP0117 based vector. The Gt::intron::maize psy::nos cassette was recovered with HindIII/Pac1 digestion and cloned into a binary vector containing a Gt::intron::SSUcrtI::nos cassette to give the construct 12421.

1.7 Maize psy (E1B)+crtI Construct 12422

The vector with the Gt::intron::maize psy::nos cassette in the pPRP0117 backbone (from construction of 12421 above) was digested with the restriction enzymes flanking the intron followed by religation of the vector in order to remove the catalase intron. The Gt::maize psy::nos cassette was recovered with HindIII/Pac1 digestion and cloned into a binary vector containing a Gt::SSUcrtI::nos cassette to give the construct 12422.

1.8 Maize psy+crtI Construct 12423

The Y1 maize psy (U32636) cds sequence was synthesised with Nco1 and Sfi1 restriction sites added at the 5′ and 3′ end respectively. This was cloned into the Nco1 and Sfi1 sites of a pPRP0117 based vector. The Gt::intron::maize psy::nos cassette was recovered with HindIII/Pac1 digestion and cloned into a binary vector containing a Gt::intron::SSUcrtI::nos cassette to give the construct 12423.

1.9 Maize psy+crtI Construct 12424

The vector with the Gt::intron::maize psy::nos cassette in the pPRP0117 backbone (from construction of 12423 above) was digested with the restriction enzymes flanking the intron followed by religation of the vector in order to remove the catalase intron. The Gt::maize psy::nos cassette was recovered with HindIII/Pac1 digestion and cloned into a binary vector containing a Gt::SSUcrtI::nos cassette to give the construct 12424.

### 2.0 Construction of Vectors for Plant Transformation.

When providing vectors for plant transformation which utilise Agrobacterium, it is preferred that the sequences according to the invention are inserted between the border regions of a single TDNA region. Agrobacterium may be transformed in accordance with methods which are well known to the person skilled in the art, and/or via the methods disclosed herein.

### 3.0 Transformation of Rice

Based on the protocol published by Hiei et al (1994 The Plant Journal, 6 (2), 271-282). The key modification involves use of a supervirulent strain in combination with a standard binary vector.

The basic procedure is as follows:

Mature rice seed (Asanohikari) are de-husked and surface sterilised by 70% ethanol for one minute followed by 4% Sodium hypochlorite+Tween for 30 minutes. Seed are then sown on a callus induction media (CIM) (N6 salts, N6 vitamins, 30 g/l sucrose, 1 g/l casein hydrolysate, 2 mg/l 2,4-D, pH 5.8, 4 g/l Gelrite) and placed in the dark at 30° C. After 3 weeks embryogenic calli are isolated and plated on CIM and placed under the same conditions. At the same time Agrobacterium cultures (AGL1 strain plus binary) are established by spreading inoculum onto LB plates plus Kanamycin (50 mg/l). After three days, Agrobacterium is scraped from the plate and re-suspended in AA1+AS (AA salts, B5 vitamins, AA Amino Acids, 68.5 g/l sucrose, 36 g/l glucose, 0.5 g/l casein hydrolysate, 100 μM Acetosyringone, pH 5.2) to an optical density of 0.1 at 600 nm. The embryogenic calli is inoculated with the Agrobacterium solution for 10 minutes after which the calli are spread on to plates of R2COMAS (R2 Micro salts, ½ R2 Macro salts, B5 Vitamins, 20 g/l sucrose, 10 g/l glucose, 1 g/l casein hydrolysate, 2 mg/l 2,4-D, 100 μM Acetosyringone, pH 5.2) and placed in the dark at 26° C. After three days calli are transferred on to selection media (N6 salts, N6 vitamins, 30 g/l sucrose, 1 g/l casein hydrolysate, 2 mg/l 2,4-D, 300 mg/l Timentin, 50 mg/l Hygromycin, 4 g/l Gelrite, pH 5.8) and place in the light at 30° C. All of the following steps occur under the same growth conditions (Light, 30° C.). After three weeks the putative transgenic calli are transferred on a pre-regeneration media (N6 salts and vitamins, 30 g/l sucrose, 1 g/l casein hydrolysate, 1 mg/l 2,4-D, 300 mg/l Timentin, 50 mg/l Hygromycin, 6 g/l Gelrite, pH 5.8). After two weeks the good quality embryogenic calli is transferred to regeneration media (N6 Micro, ½ N6 Macro, N6 vitamins, AA amino acids, 20 g/l sucrose, 1 g/l casein hydrolysate, 0.2 mg/l NAA, 1 mg/l Kinetin, 50 mg/l Hygromycin, pH 5.8, Gelrite 6 g/l). After three weeks, plantlets that regenerate are subcultured to rooting media (½ MS salts, ½ B5 Vitamins, 10 g/l sucrose, 25 mg/l Hygromycin, pH 5.8, 8 g/l microagar. After two weeks the plantlets that have robust root systems are transferred to soil (50% John Innes #3, 50% peat, 26° C., 16 hour photoperiod) and covered with a fleece until the plants are established.

### 4.0 Analysis of Carotenoids in Rice Transformants

Carotenoids are extracted from seeds harvested at maturity. Seed is dehusked using a TR-200 Electromotion Rice Husker and then polished for 1 min. with a Pearlest polisher (Kett). Any white or discoloured seed are removed and 0.5 g of the sample is ground for 2 minutes using a Glen Creston 8000 Mixer/Mill. A total of 1 g of ground material/plant may be obtained by this method. This can be thoroughly mixed together prior to extraction of 2×0.5 g portions of the powder. A standard compound can then be added to the samples for quantification of recoveries. Astaxanthin and echinenone are examples of standards which can be used. Samples are hydrated with 1 ml of water and mixed using a vortex for a few seconds. 6 ml of acetone is then added and the samples sonicated for 2 minutes. The samples are centrifuged for 5 min. at 3500 rpm. The supernatants are decanted, and the samples re-extracted twice more with 3 ml acetone, repeating the sonication/centrifugation steps between extractions. One extraction with 2 ml of tert-methylbutylether is then performed including the sonication/centrifugation steps and all supernatants for one sample pooled together. The total volume for each extract is adjusted to 14 ml with acetone and the centrifugation step repeated. A 2 ml aliquot of each sample is evaporated to dryness under a stream of nitrogen gas. These aliquots are re-dissolved in 75 μl of ethyl acetate, vortexed for 5-10 s and then transferred to amber HPLC insert vials. The vials are sealed immediately and centrifuged again at 3500 rpm prior to HPLC analysis.

The HPLC quantification is based on the response factor determined for each reference standards. The overall concentration of the prepared and dissolved reference standards is measured spectrophotometrically based on the published molar extinction coefficients and/or absorbance measured for solution of 1% concentration using 1 cm path length (A 1%1 cm) (Ref: Britton G., Liaanen-Jensen S. and Pfander H. P. (1995) Carotenoids: Spectroscopy Vol 1B pp 57-62, Birkhauser Verlag, Basel, ISBN 3-7643-2909-2). For each standard stock solution, the purity of the principal component is determined by HPLC. For components where no reference standard is available e.g. various cis isomers quantitative results are expressed using the response factor for that of β-carotene.

### 4.1 HPLC Equipment & Conditions

| | |
|---|---|
| Pump: | Agilent 1100 Quaternary or Binary Pump; |
| | model number G1311A or G1312A, |
| | respectively |
| Degasser: | Agilent 1100 Degasser; model number G1322A |
| Temperature Controlled | Agilent 1100 Automatic Liquid Sampler; model |
| Autosampler: | number G1313A equipped with autosampler |
| | temperature controller model number G1330A |
| Detector: | Agilent 1100 Diode array detector; model |
| | number G1315A or G1315B |
| | Agilent 1100 fluorescence detector; model |
| | number G1321A (for tocopherol analysis only). |
| Column Oven: | Agilent 1100 Column Compartment; model |
| | number G1316A |
| Instrument Conditions | |
| Column: | YMC C30 3 μm particles in 25 cm × 4.6 mm id |
| | stainless steel column + 1 cm × 4 mm 5 μm |
| | YMC C30 guard column |
| Column temperature: | 25° C. |
| Sample temperature: | 4° C. |
| Mobile phase: | Solvent A = MeOH/H2O/tert-butylmethylether |
| | (TBME) + 1.3 mM NH4acetate (70/25/5 v/v) |
| | Solvent B = MeOH/H2O/TBME + 1.3 mM |
| | NH4acetate (7/3/90 v/v) |
| Stop time | 30 min |
| Post time | 0 min |
| Flow rate | 1 ml min−1 |
| Injection volume | 25 μl in ethyl acetate |

Gradient Conditions (6%/min):

| | A % | B % |
|---|---|---|
| | MeOH/H2O/TBME + | MeOH/H2O/TBME + |
| | 1.3 mM NH4acetate | 1.3 mM NH4acetate |
| Time (min) | (70/25/5 v/v) | (7/3/90 v/v) |
| 0 | 95 | 5 |
| 15.83 | 0 | 100 |
| 22 | 0 | 100 |
| 24 | 95 | 5 |
| 30 | 95 | 5 |

### 5.0 Results of HPLC Quantification of Carotenoids:

Results for rice plants transformed with construct 11586 (construct described in 1.5 above)

| Sample | μg/g dry weight (dwt) |
|---|---|
| identity | endosperm |
| wild type | 0.05 |
| 11586-10 | 4.19 |
| 11586-7 | 6.11 |
| 11586-25 | 6.32 |
| 11586-15 | 7.55 |
| 11586-30 | 7.69 |
| 11586-28 | 9.05 |
| 11586-14 | 11.82 |
| 11586-20 | 12.82 |
| 11586-1 | 13.29 |
| 11586-12 | 18.59 |

| Sequence ID Number | Components | μg/g dwt endosperm |
|---|---|---|
| 7 | crtI + pepper psy | >3 |
| 8 | crtI + tomato psy | >3 |
| 9 | crtI + crtB | >5 |
| — | Untransformed Control | 0 |

6.0 Transformation of Rice—Method Used for Transformation of Rice with Agrobacterium comprising constructs 12421, 12422, 12423 and 12424 (constructs described in examples 1.6, 1.7, 1.8 and 1.9 respectively).

For this example, rice (Oryza sativa) is used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in concentration or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium strain LBA4404 containing the desired vector construction. Agrobacterium is cultured from glycerol stocks on solid YPC medium (100 mg/L spectinomycin and any other appropriate antibiotic) for ˜2 days at 28° C. Agrobacterium is re-suspended in liquid MS-CIM medium. The Agrobacterium culture is diluted to an OD600 of 0.2-0.3 and acetosyringone is added to a final concentration of 200 uM. Agrobacterium is induced with acetosyringone before mixing the solution with the rice cultures. For inoculation, the cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7-1 medium (MS with no hormones and 2% Sorbitol) for 2 weeks and then moved to the greenhouse when they are large enough and have adequate roots. Plants are transplanted to soil in the greenhouse and grown to maturity.

7.0 Method of Extraction/Quantification of Carotenoids—for Plants Transformed in Accordance with Example 6.0
(a) Sample is grinded in a Geno/Grinder at 1600 rpm for 40 seconds.
(b) Representative amounts of homogenised sample (0.1 g) are weighed into an appropriate extraction vessel (e.g. 2 ml microcentrifuge tube). Sample weight is recorded.
(c) Samples are hydrated with water (200 μl) and vortexed for about 2-3 seconds then left to stand for about 10 minutes.
(d) Acetone (1.2 ml) is added to the sample.
(e) Ultrasonicate sample for 5 minutes.
(f) Centrifuge sample for 3 min at 6000 rpm, then transfer the supernatant to another appropriately sized tube.
(g) Steps (d) to (f) are repeated and combined with the previous extract.
(h) Steps (d) to (f) are repeated with tert-butylmethylether (400 μl) and combine with the acetone extracts.
(i) All of the combined extracts are transferred into an appropriate size tube and evaporate it to dryness under a stream of nitrogen gas.
(j) Samples are redissolved in 2 ml of ethyl acetate+0.5% BHT, vortex or ultrasonicate for 5-10 seconds and then centrifuged at 3500 rpm for 5 minutes before HPLC analysis.
(k) Absorption at wavelength 450 nm is measured on a spectrophotometer for all samples and total carotenoid concentration is calculated based on an 8 value of 124865.

### 8.0 Results of the Method of Example 7.0

| | | Calculated |
|---|---|---|
| Construct | Sample ID | Carotenoids (μg/g) |
| 12421 | RIGQ2003001046A4A | 48.7 |
| 12421 | RIGQ2003001063A43A | 42.7 |
| 12421 | RIGQ2003001063A4A | 36.9 |
| 12421 | RIGQ2003001063A99A | 31.4 |
| 12421 | RIGQ2003001063A59A | 30 |
| 12421 | RIGQ2003001063A75A | 28.7 |
| 12421 | RIGQ2003001048A3A | 28.5 |
| 12421 | RIGQ2003001050A23A | 28 |
| 12421 | RIGQ2003001050A13A | 26.8 |
| 12421 | RIGQ2003001048A25A | 26.8 |
| 12421 | RIGQ2003001048A61A | 26.1 |
| 12421 | RIGQ2003001049A46A | 23.4 |
| 12421 | RIGQ2003000993A63A | 21.7 |
| 12421 | RIGQ2003001049A43A | 20.6 |
| 12421 | RIGQ2003001049A26A | 10.5 |
| 12421 | RIGQ2003001050A18A | 5.7 |
| 12422 | RIGQ2003001045A30A | 58.8 |
| 12422 | RIGQ2003001045A51A | 54 |
| 12422 | RIGQ2003000995A29A | 43.6 |
| 12422 | RIGQ2003000995A31A | 43.1 |
| 12422 | RIGQ2003001043A10A | 42.4 |
| 12422 | RIGQ2003001043A8A | 42 |
| 12422 | RIGQ2003000995A19A | 41.9 |
| 12422 | RIGQ2003000995A17A | 40.9 |
| 12422 | RIGQ2003001060A23A | 37.2 |
| 12422 | RIGQ2003001052A56A | 35 |
| 12422 | RIGQ2003001051A75A | 32 |
| 12422 | RIGQ2003001045A68A | 30.6 |
| 12422 | RIGQ2003001045A86A | 29.8 |
| 12422 | RIGQ2003001045A49A | 29.2 |
| 12422 | RIGQ2003001060A25A | 28.9 |
| 12422 | RIGQ2003001051A64A | 26.4 |
| 12422 | RIGQ2003001051A34A | 25.2 |
| 12422 | RIGQ2003000994A7A | 22.8 |
| 12422 | RIGQ2003001045A74A | 20.3 |
| 12422 | RIGQ2003001051A46A | 16.6 |
| 12422 | RIGQ2003000995A7A | 13.8 |
| 12422 | RIGQ2003000995A26A | 8.1 |
| 12422 | RIGQ2003000995A41A | 6.8 |
| 12422 | RIGQ2003000995A8A | 3.8 |
| 12423 | RIGQ2003001097A42A | 52.80 |
| 12423 | RIGQ2003001097A15A | 50.40 |
| 12423 | RIGQ2003001097A41A | 48.00 |
| 12423 | RIGQ2003001114A10A | 44.20 |
| 12423 | RIGQ2003001099A8A | 40.00 |
| 12423 | RIGQ2003001098A2A | 28.80 |
| 12423 | RIGQ2003001099A42A | 26.00 |
| 12423 | RIGQ2003001097A30A | 24.00 |
| 12423 | RIGQ2003001186A80A | 23.30 |
| 12423 | RIGQ2003001097A18A | 21.60 |
| 12423 | RIGQ2003001099A60A | 17.60 |
| 12423 | RIGQ2003001098A5A | 17.40 |
| 12423 | RIGQ2003001097A44A | 12.00 |
| 12424 | RIGQ2003001121A60A | 51.9 |
| 12424 | RIGQ2003001121A20A | 51.8 |
| 12424 | RIGQ2003001093A11A | 40 |
| 12424 | RIGQ2003001094A85A | 37.8 |
| 12424 | RIGQ2003001093A58A | 27.3 |
| 12424 | RIGQ2003001118A84A | 26 |

Although the invention has been described by way of the above-referenced examples and the Sequence Listing and Figures as provided herein, it will be apparent that modifications and changes may be practiced which remain within the ambit of the present invention.

### REFERENCES

| | Database | | |
|---|---|---|---|
| Name | Accession No. | Description | Reference |
| Glutelin1 | NCBI | Rice, promoter | Takaiwa, F.; Ebinuma, H.; Kikuchi, S.; |
| | D00584, | of a rice seed | Oono, K.; |
| | EMBL | storage protein | Nucleotide sequence of a rice glutelin gene, |
| | Y00867 | | FEBS Lett. 221: 43 (1987) |
| Prolamin | D73383 | Rice, promoter | Nakase, M.; Yamada, T.; Kira, T.; |
| | D73384 | of a rice seed | Yamaguchi, J.; Aoki, N.; Nakamura, R.; |
| | | storage protein | Matsuda, T.; Adachi, T.. The same nuclear |
| | | | proteins bind to the 5′-flanking regions of |
| | | | genes for the rice seed storage protein: 16 kDa |
| | | | albumin, 13 kDa prolamin and type II |
| | | | glutelin. |
| | | | Plant Mol. Biol. 32: 621-630 (1996) |
| Prolamin | M23746 | Rice, promoter | Kim, W. T.; Okita, T. W.; Structure, expression, |
| | | of a rice seed | and heterogeneity of the rice seed prolamines |
| | | storage protein | Plant Physiol. 88: 649 (1988) |
| 1st intron | D21161 | Castor bean | Suzuki, M.; Ario, T.; Hattori, T.; Nakamura, K.; |
| of catalase | | | Isolation and characterization of two tightly |
| gene | | | linked catalase genes from castor bean that are |
| | | | differentially regulated Plant Mol. Biol. |
| | | | 25: 507 (1994) |
| SSU | X00806 | Pea, signal | Coruzzi, G.; Broglie, R.; Edwards, C.; |
| | | peptide of small | Chua, N. H.; Tissue-specific and light- |
| | | subunit of | regulated expression of a pea nuclear gene |
| | | rubisco | encoding the small subunit of ribulose-1,5- |
| | | | bisphosphate carboxylase EMBO J. 3: 1671 |
| | | | (1984) |
| SSU | X04334 | Pea, signal | Fluhr, R.; Moses, P.; Morelli, G.; Coruzzi, G.; |
| | | peptide of small | Chua, N. H.; Expression dynamics of the pea |
| | | subunit of | rbcS multigene family and organ distribution |
| | | ribulose | of the transcripts EMBO J. 5: 2063 (1986) |
| | | bisphosphate | |
| | | carboxylase | |
| Maize psy | U32636 | Maize phytoene | Buckner, B.; Miguel, P. S.; Janick-Buckner, D.; |
| | | synthase gene | Bennetzen, J. L.; The y1 gene of maize codes |
| | | | for phytoene synthase Genetics 143(1): 479 |
| | | | (1996) |
| Pepper | X68017 | Pepper | Romer, S.; Hugueney, P.; Bouvier, F.; |
| psy | | phytoene | Camara, B.; Kuntz, M.; Expression of the |
| | | synthase gene | genes encoding the early carotenoid |
| | | | biosynthetic enzymes in Capsicum annuum |
| | | | Biochem. Biophys. Res. Commun. 196: 1414 |
| | | | (1993) |
| Tomato | Y00521 | tomato | Ray, J.; Bird, C. R.; Maunders, M.; Grierson, D.; |
| psy | | phytoene | Schuch, W.; Sequence of ptom 5 a ripening |
| | | synthase gene | related cDNA from tomato Nucleic Acids |
| | | | Res. 15: 10587 (1987) |
| Daffodil | X78814 | daffodil | Schledz, M.; Ali Babili, S.; Lintig, J.; |
| psy | | phytoene | Haubruck, H.; Rabbani, S.; Kleing, H.; |
| | | synthase gene | Beyer, P.; Phytoene synthase from Narcissus |
| | | | pseudonarcissus: functional, expression, |
| | | | galactolipid requirement, topological |
| | | | distribution in chromoplasts and induction |
| | | | during flowering Plant J. 10: 781 (1996) |
| CrtB | D90087 | Erwinia | Misawa, N.; Nakagawa, M.; Kobayashi, K.; |
| | | uredovora | Yamano, S.; Izawa, Y.; Nakamura, K.; |
| | | phytoene | Harashima, K.; Elucidation of the Erwinia |
| | | synthase | uredovora carotenoid biosynthetic pathway by |
| | | | functional analysis of gene products expressed |
| | | | in Escherichia coli J. Bacteriol. 172: 6704 |
| | | | (1990) |
| CrtI | D90087 | Erwinia | Misawa, N.; Nakagawa, M.; Kobayashi, K.; |
| | | uredovora | Yamano, S.; Izawa, Y.; Nakamura, K.; |
| | | phytoene | Harashima, K.; Elucidation of the Erwinia |
| | | desaturase | uredovora carotenoid biosynthetic pathway by |
| | | | functional analysis of gene products expressed |
| | | | in Escherichia coli J. Bacteriol. 172: 6704 |
| | | | (1990) |
| Nos | AJ237588 | Agrobacterium | |
| | | tumefaciens Ti | |
| | | plasmid | |
| Phytoene | M88683 | Tomato | Giuliano, G.; Bartley, G. E.; Scolnik, P. A.; |
| Desaturase | | | Regulation of carotenoid biosynthesis during |
| | | | tomato development Plant Cell 5(4): 379 |
| | | | (1993) |
| Phytoene | X68058 | Pepper | Hugueney, P.; Roemer, S.; Kuntz, M.; |
| Desaturase | | | Camara, B.; Characterization and molecular |
| | | | cloning of a flavoprotein catalyzing the |
| | | | synthesis of phytofluenen and zeta-carotene in |
| | | | Capsicum chromoplasts Eur. J. Biochem. |
| | | | 209: 399 (1992) |
| Phytoene | U37285 | Maize | Li, Z. H.; Matthews, P. D.; Burr, B.; |
| Desaturase | | | Wurtzel, E. T.; Cloning and characterization of |
| | | | a maize cDNA encoding phytoene desaturase, |
| | | | an enzyme of the carotenoid biosynthetic |
| | | | pathway Plant Mol. Biol. 30(2): 269 (1996) |
| Phytoene | AF049356 | Rice | Vigneswaran, A.; Wurtzel, E. T.; A Rice cDNA |
| Desaturase | | | Encoding Phytoene Desaturase (Accession |
| | | | No. AF049356) (PGR99-131) Plant Physiol. |
| | | | 121(1): 312 (1999) |
| ZDS | AF195507 | Tomato | Bartley, G. E.; Ishida, B. K.; Zeta-carotene |
| | | | desaturase (Accession No. AF195507) from |
| | | | tomato (PGR99-181) Plant Physiol. |
| | | | 121(4): 1383 (1999) |
| ZDS | X89897 | Pepper | Albrecht, M.; Klein, A.; Hugueney, P.; |
| | | | Sandmann, G.; Kuntz, M.; Molecular cloning |
| | | | and functional expression in E. coli of a novel |
| | | | plant enzyme mediating zeta-carotene |
| | | | desaturation FEBS Lett. 372: 199 (1995) |
| ZDS | AF047490 | Maize | Luo, R.; Wurtzel, E. T.; A Maize cDNA |
| | | | Encoding Zeta Carotene Desaturase |
| | | | (Accession No. AF047490). (PGR99-118) |
| | | | Plant Physiol. 120(4): 1206 (1999) |
| ZDS | AF054629 | Rice | |

That which is claimed:

1. A polynucleotide comprising:
(a) a region which comprises as operably linked components (i) a promoter which provides for seed preferred expression; (ii) a nucleotide sequence derived from a bacterium which sequence encodes a carotene desaturase; and (iii) a transcription termination region; and
(b) a further region which comprises as operably linked components (i) a promoter which provides for seed preferred expression; (ii) a nucleotide sequence encoding a phytoene synthase which sequence is derived from maize (Zea sp.) or rice (Orzya sp.); and (iii) a transcription termination region.

2. A polynucleotide according to claim 1, wherein the sequence which encodes the carotene desaturase is derived from Erwinia sp.

3. A polynucleotide according to claim 1, wherein said promoter is selected from the group consisting of: Glutelin 1 and Prolamin, and wherein said transcription termination region is selected from the group consisting of: Nos, CaMV 35S, and PotP1-II.

4. A polynucleotide according to claim 1, wherein the sequence which encodes carotene desaturase and the sequence which encodes phytoene synthase further comprises a sequence encoding a plastid targeting sequence.

5. A polynucleotide according to claim 1, wherein said polynucleotide comprises a nucleotide sequence selected from the group consisting of: SEQ ID NO: 1, SEQ ID NO: 2, SEQ ID NO: 3, SEQ ID NO: 4, and SEQ ID NO: 6.

6. A polynucleotide sequence which is the complement of one which hybridises to a polynucleotide according to claim 5 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still comprises a region encoding a carotene desaturase and a further region encoding a phytoene synthase and wherein when said polynucleotide sequence is inserted into plant material the seed of a plant regenerated from said material produces carotenoids at a level of at least 10 μg/g of endosperm of said seed.

7. A polynucleotide sequence which is the complement of one which hybridises to a polynucleotide according to claim 5 at a temperature of about 65° C. in a solution containing 6×SSC, 0.01% SDS and 0.25% skimmed milk powder, followed by rinsing at the same temperature in a solution containing 0.2×SSC and 0.1% SDS wherein said polynucleotide sequence still comprises a region encoding a carotene desaturase and a further region encoding a phytoene synthase and when said polynucleotide sequence is inserted into plant material the seed of a plant regenerated from said material produce carotenoids amounting to at least 80% of the carotenoid content of a seed which comprises a polynucleotide selected from the group consisting of SEQ ID NO: 1, SEQ ID NO: 2, SEQ ID NO: 3, SEQ ID NO: 4, and SEQ ID NO: 6.

8. A polynucleotide sequence according to claim 6 wherein said seed is a rice seed.

9. A polynucleotide according to claim 1 which further comprises a region which encodes a selectable marker.

10. A polynucleotide according to claim 9, wherein said selectable marker comprises a mannose-6-phosphate isomerase gene.

11. A polynucleotide according to claim 1 which is codon optimised for expression in a particular plant species.

12. A polynucleotide sequence according to claim 11, wherein said plant species is rice (Orzya sp.).

13. A method for increasing the carotenoid content of seeds comprising inserting into plant material a polynucleotide according to claim 1; and regenerating a seed-containing plant from said material and identifying the seeds which contain carotenoids at levels greater than those of control like-seeds.

14. A method for increasing the carotenoid content of a seed comprising inserting into plant material a polynucleotide comprising a sequence selected from the group consisting of: SEQ ID NO: 1, SEQ ID NO: 2, SEQ ID NO: 3, SEQ ID NO: 4, and SEQ ID NO: 6; and regenerating a seed-containing plant from said material and identifying the seed which contains carotenoids at levels greater than those of a control like-seed.

15. A method according to claim 13, wherein said seed contains at least a sixty fold increase in carotenoids when compared to a control like-seed.

16. A method according to claim 13, wherein said seed contains carotenoids at a level of at least 10 μg/g of endosperm of said seed.

17. A method according to claim 13, wherein said carotenoids are selected from the group consisting of: lycopene; alpha-carotene; lutein; beta-carotene; zeanthin; antheraxanthin; violaxanthin; and neoxanthin or a combination thereof.

18. A plant or plant material which comprises a polynucleotide according to claim 1.

19. A plant or plant material according to claim 18 which is a rice plant or is rice plant material.

20. A plant or plant material according to claim 18 which is a maize plant or is maize plant material.


SEQUENCE LISTING (38 sequences)

SEQ ID NO: 1 - DNA, 5630 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaataagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 2 - DNA, 5630 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaataagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtaccagtgatgggcatcgcatccgagtctaaagcaacaactgaaagcgtgtacagtgctgccttggctctcggaattgcgaaccaactcacgaacatactccgggatgttggagaggatgctagacgaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 3 - DNA, 5180 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaataagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccaaatcgccgccaccatggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtaccagtgatgggcatcgcatccgagtctaaagcaacaactgaaagcgtgtacagtgctgccttggctctcggaattgcgaaccaactcacgaacatactccgggatgttggagaggatgctagacgaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 4 - DNA, 5180 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaataagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccaaatcgccgccaccatggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 5 - DNA, 5653 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaatcgcaagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaaatgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 6 - DNA, 5714 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaaaactgaaggcgggaaacgacaatctgatctctaggaagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcggccatcacgctcctacgttcagcgtctcttccgggcctctccgacgccctcgcccgggacgctgctgccgtccaacatgtctgctcctcctacctgcccaacaacaaggagaagaagaggaggtggatcctctgctcgctcaagtacgcctgccttggcgtcgaccctgccccgggcgagattgcccggacctcgccggtgtactccagcctcaccgtcacccctgctggagaggccgtcatctcctcggagcagaaggtgtacgacgtcgtcctcaagcaggcagcattgctcaaacgccacctgcgcccacaaccacacaccattcccatcgttcccaaggacctggacctgccaagaaacggcctcaagcaggcctatcatcgctgcggagagatctgcgaggagtatgccaagaccttttaccttggaactatgctcatgacggaggaccgacggcgcgccatatgggccatctatgtgtggtgtaggaggacagatgagcttgtagatggaccaaatgcctcgcacatcacaccgtcagccctggaccggtgggagaagaggcttgatgatctcttcaccggacgcccctacgacatgcttgatgctgcactttctgataccatctccaagtttcctatagatattcagcctttcagggacatgatagaagggatgcggtcagacctcagaaagactagatacaagaacttcgacgagctctacatgtactgctactatgttgctggaactgtggggctaatgagtgttcctgtgatgggtattgcacccgagtcgaaggcaacaactgaaagtgtgtacagtgctgctttggctctcggcattgcaaaccagctcacaaatatactccgtgacgttggagaggacgcgagaagagggaggatatatttaccacaagatgaacttgcagaggcagggctctctgatgaggacatcttcaatggcgttgtgactaacaaatggagaagcttcatgaagagacagatcaagagagctaggatgttttttgaggaggcagagagaggggtgaccgagctcagccaggcaagccggtggccggtctgggcgtctctgttgttataccggcaaatccttgacgagatagaagcaaacgattacaacaacttcacaaagagggcgtacgttgggaaggcgaagaaattgctagcgcttccagttgcatatggtagatcattgctgatgccctactcactgagaaatagccagaagtagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 7 - DNA, 5974 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaatgttcggggcgaacatcgcaagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatgtctgttgccttgttatgggttgtttctccttgtgacgtctcaaacgggacaggattcttggtatccgttcgtgagggaaaccggatttttgattcgtcggggcgtaggaatttggcgtgcaatgagagaatcaagagaggaggtggaaaacaaaggtggagttttggttcttacttgggaggagcacaaactggaagtggacggaaattttctgtacgttctgctatcgtggctactccggctggagaaatgacgatgtcatcagaacggatggtatatgatgtggttttgaggcaggcagccttggtgaagagacagctgagatcgaccgatgagttagatgtgaagaaggatatacctattccggggactttgggcttgttgagtgaagcatatgataggtgtagtgaagtatgtgcagagtacgcaaagacgttttacttaggaacgatgctaatgactccggagagaagaaaggctatctgggcaatatacgtatggtgcaggagaacagacgaacttgttgatggtccgaatgcatcacacattactccggcggccttagataggtgggaagacaggctagaagatgttttcagtggacggccatttgacatgctcgatgctgctttgtccgacacagtttccaaatttccagttgatattcagccattcagagatatgattgaaggaatgcgtatggacttgaggaagtcaagatacagaaactttgacgaactatacctatattgttattacgttgctggtacggttgggttgatgagtgttccaattatgggcatcgcacctgaatcaaaggcaacaacggagagcgtatataatgctgctttggctttggggatcgcaaatcagctgaccaacatacttagagatgttggagaagatgccagaagaggaagagtctatttgcctcaagatgaattagcacaggcaggtctatccgacgaagacatatttgctggaagagtgaccgataaatggagaatcttcatgaagaaacaaattcagagggcaagaaagttctttgacgaggcagagaaaggagtgaccgaattgagcgcagctagtagatggcctgtgttggcatctctgctgttgtaccgcaggatactggacgagatcgaagccaatgactacaacaacttcacaaagagagcttatgtgagcaaaccaaagaagttgattgcattacctattgcatatgcaaaatctcttgtgccttctacaagaacatgaaatcaggattttatataaatcaaggccaatgaagccaatatacatttagaagaaaaaaaacaagtgtttataaagtagaattattgaaggggaggcttggagtaactggtaaagttgttgtcatgtgactgggaagtcacgggttcaagccttggaaacagcctctggcagaaatgcaaggtaaggttgcgtacaatataccgttaaggtggggtccttcccagtacaccgcgcatagcgatagatttagtgcaccgggtcgccttttttctaaagtagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 8 - DNA, 5782 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaatcgcaagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggaactatgctaatgactcccgagagaagaagggctatctgggcaatatatgtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcagccattcagagatatgattgaaggaatgcgtatggacttgagaaaatcgagatacaaaaacttcgacgaactatacctttattgttattatgttgctggtacggttgggttgatgagtgttccaattatgggtatcgcccctgaatcaaaggcaacaacagagagcgtatataatgctgctttggctctggggatcgcaaatcaattaactaacatactcagagatgttggagaagatgccagaagaggaagagtctacttgcctcaagatgaattagcacaggcaggtctatccgatgaagatatatttgctggaagggtgaccgataaatggagaatctttatgaagaaacaaatacatagggcaagaaagttctttgatgaggcagagaaaggcgtgacagaattgagctcagctagtagattccctgtatgggcatctttggtcttgtaccgcaaaatactagatgagattgaagccaatgactacaacaacttcacaaagagagcatatgtgagcaaatcaaagaagttgattgcattacctattgcatatgcaaaatctcttgtgcctcctacaaaaactgcctctcttcaaagataaagcatgaaatgaagatatatatatatatatatatagcaatatacattagaagaaaaaaaggaagaagaaatgttgttgtattgatataaatgtatatcataaatattaggttgtagtaacattggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 9 - DNA, 5551 bp, Artificial Sequence
  1-839: Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaatcgcaagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacgaattcggcttcccgggtacagggtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacagaaccgtcgactctagagaagccatttaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgcgatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgacgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctagggatccgttaagggcgaattccagcacactggcggccgttactagtggatccgagctcggtacctcgacggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 10 - DNA, 1233 bp, Zea mays
atggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 11 - DNA, 1233 bp, Zea mays
atggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaaatgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 12 - DNA, 1233 bp, Zea mays
atggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtaccagtgatgggcatcgcatccgagtctaaagcaacaactgaaagcgtgtacagtgctgccttggctctcggaattgcgaaccaactcacgaacatactccgggatgttggagaggatgctagacgaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 13 - DNA, 1263 bp, Oryza sp.
atggcggccatcacgctcctacgttcagcgtctcttccgggcctctccgacgccctcgcccgggacgctgctgccgtccaacatgtctgctcctcctacctgcccaacaacaaggagaagaagaggaggtggatcctctgctcgctcaagtacgcctgccttggcgtcgaccctgccccgggcgagattgcccggacctcgccggtgtactccagcctcaccgtcacccctgctggagaggccgtcatctcctcggagcagaaggtgtacgacgtcgtcctcaagcaggcagcattgctcaaacgccacctgcgcccacaaccacacaccattcccatcgttcccaaggacctggacctgccaagaaacggcctcaagcaggcctatcatcgctgcggagagatctgcgaggagtatgccaagaccttttaccttggaactatgctcatgacggaggaccgacggcgcgccatatgggccatctatgtgtggtgtaggaggacagatgagcttgtagatggaccaaatgcctcgcacatcacaccgtcagccctggaccggtgggagaagaggcttgatgatctcttcaccggacgcccctacgacatgcttgatgctgcactttctgataccatctccaagtttcctatagatattcagcctttcagggacatgatagaagggatgcggtcagacctcagaaagactagatacaagaacttcgacgagctctacatgtactgctactatgttgctggaactgtggggctaatgagtgttcctgtgatgggtattgcacccgagtcgaaggcaacaactgaaagtgtgtacagtgctgctttggctctcggcattgcaaaccagctcacaaatatactccgtgacgttggagaggacgcgagaagagggaggatatatttaccacaagatgaacttgcagaggcagggctctctgatgaggacatcttcaatggcgttgtgactaacaaatggagaagcttcatgaagagacagatcaagagagctaggatgttttttgaggaggcagagagaggggtgaccgagctcagccaggcaagccggtggccggtctgggcgtctctgttgttataccggcaaatccttgacgagatagaagcaaacgattacaacaacttcacaaagagggcgtacgttgggaaggcgaagaaattgctagcgcttccagttgcatatggtagatcattgctgatgccctactcactgagaaatagccagaagtag

SEQ ID NO: 14 - protein, 420 aa, Oryza sp.
MAAITLLRSASLPGLSDALARDAAAVQHVCSSYLPNNKEKKRRWILCSLKYACLGVDPAPGEIARTSPVYSSLTVTPAGEAVISSEQKVYDVVLKQAALLKRHLRPQPHTIPIVPKDLDLPRNGLKQAYHRCGEICEEYAKTFYLGTMLMTEDRRRAIWAIYVWCRRTDELVDGPNASHITPSALDRWEKRLDDLFTGRPYDMLDAALSDTISKFPIDIQPFRDMIEGMRSDLRKTRYKNFDELYMYCYYVAGTVGLMSVPVMGIAPESKATTESVYSAALALGIANQLTNILRDVGEDARRGRIYLPQDELAEAGLSDEDIFNGVVTNKWRSFMKRQIKRARMFFEEAERGVTELSQASRWPVWASLLLYRQILDEIEANDYNNFTKRAYVGKAKKLLALPVAYGRSLLMPYSLRNSQK

SEQ ID NO: 15 - DNA, 1260 bp, Capsicum annuum
atgtctgttgccttgttatgggttgtttctccttgtgacgtctcaaacgggacaggattcttggtatccgttcgtgagggaaaccggatttttgattcgtcggggcgtaggaatttggcgtgcaatgagagaatcaagagaggaggtggaaaacaaaggtggagttttggttcttacttgggaggagcacaaactggaagtggacggaaattttctgtacgttctgctatcgtggctactccggctggagaaatgacgatgtcatcagaacggatggtatatgatgtggttttgaggcaggcagccttggtgaagagacagctgagatcgaccgatgagttagatgtgaagaaggatatacctattccggggactttgggcttgttgagtgaagcatatgataggtgtagtgaagtatgtgcagagtacgcaaagacgttttacttaggaacgatgctaatgactccggagagaagaaaggctatctgggcaatatacgtatggtgcaggagaacagacgaacttgttgatggtccgaatgcatcacacattactccggcggccttagataggtgggaagacaggctagaagatgttttcagtggacggccatttgacatgctcgatgctgctttgtccgacacagtttccaaatttccagttgatattcagccattcagagatatgattgaaggaatgcgtatggacttgaggaagtcaagatacagaaactttgacgaactatacctatattgttattacgttgctggtacggttgggttgatgagtgttccaattatgggcatcgcacctgaatcaaaggcaacaacggagagcgtatataatgctgctttggctttggggatcgcaaatcagctgaccaacatacttagagatgttggagaagatgccagaagaggaagagtctatttgcctcaagatgaattagcacaggcaggtctatccgacgaagacatatttgctggaagagtgaccgataaatggagaatcttcatgaagaaacaaattcagagggcaagaaagttctttgacgaggcagagaaaggagtgaccgaattgagcgcagctagtagatggcctgtgttggcatctctgctgttgtaccgcaggatactggacgagatcgaagccaatgactacaacaacttcacaaagagagcttatgtgagcaaaccaaagaagttgattgcattacctattgcatatgcaaaatctcttgtgccttctacaagaacatga

SEQ ID NO: 16 - DNA, 1239 bp, Lycopersicon esculentum
atgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggaactatgctaatgactcccgagagaagaagggctatctgggcaatatatgtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcagccattcagagatatgattgaaggaatgcgtatggacttgagaaaatcgagatacaaaaacttcgacgaactatacctttattgttattatgttgctggtacggttgggttgatgagtgttccaattatgggtatcgcccctgaatcaaaggcaacaacagagagcgtatataatgctgctttggctctggggatcgcaaatcaattaactaacatactcagagatgttggagaagatgccagaagaggaagagtctacttgcctcaagatgaattagcacaggcaggtctatccgatgaagatatatttgctggaagggtgaccgataaatggagaatctttatgaagaaacaaatacatagggcaagaaagttctttgatgaggcagagaaaggcgtgacagaattgagctcagctagtagattccctgtatgggcatctttggtcttgtaccgcaaaatactagatgagattgaagccaatgactacaacaacttcacaaagagagcatatgtgagcaaatcaaagaagttgattgcattacctattgcatatgcaaaatctcttgtgcctcctacaaaaactgcctctcttcaaagataa

SEQ ID NO: 17 - DNA, 891 bp, Erwinia sp.
atggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgcgatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgacgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctag

SEQ ID NO: 18 - DNA, 1479 bp, Erwinia sp.
atgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttga

SEQ ID NO: 19 - DNA, 1488 bp, Erwinia sp.
atggcggccgccaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttga

SEQ ID NO: 20 - DNA, 839 bp, Oryza sp.
gttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaac

SEQ ID NO: 21 - DNA, 642 bp, Oryza sp.
aagcttgcgcgcggaatacggtggagatgggttgggaaccctggattccaaacacagcccaagtctatccaaaatgtttagacaagaaaatacgtaacaagttggtttacagaaatacgaattagatcaatcctgcactacaagtagagtaaagtggtgatttctcttaaatctctcgaatggtgatttaagaattcagtgcaaaccaaatccttgctataatcaaatgttcggtaccgcatcaacggaacaataaaaagcgcctggcgtaccataattttgtcattcttgttgaaatttgtaatttaagatgcatgaggccacacgaccttaatgttcaacgtgtcatgcattagtgaaataatagctcacaaaacgcaacaaatatagctagataacggttgcaatccttaccaaactaacgtataaagtgagcgatgagtcatatcattatctcccgcctgctaaccatcgtgtacaccatccgatcacaaaaatgacaacttctagggatgaacctggacaaggtttagggtttagggatgaatctggacaaatgattgttcaggttcatccctagatgttggtttctcctgacgggacggagggagtatatgtgatggacacaaaagttactttcat

SEQ ID NO: 22 - DNA, 190 bp, Artificial Sequence
  Intron
gtaaatttctagtttttctccttcattttcttggttaggacccttttctctttttatttttttgagctttgatctttctttaaactgatctattttttaattgattggttatcgtgtaaatattacatagctttaactgataatctgattactttatttcgtgtgtctttgatcatcttgatagttacag

SEQ ID NO: 23 - DNA, 171 bp, Pisum sativum
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgc

SEQ ID NO: 24 - DNA, 254 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 25 - DNA, 193 bp, Cauliflower mosaic virus
gctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccag

SEQ ID NO: 26 - DNA, 238 bp, Solanum tuberosum
ccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatac

SEQ ID NO: 27 - DNA, 2321 bp, Lycopersicon esculentum
gggtttatctcgcaagtgtggctatggtgggacgtgtcaaattttggattgtagccaaacatgagatttgatttaaagggaattggccaaatcaccgaaagcaggcatcttcatcataaattagtttgtttatttatacagaattatacgcttttactagttatagcattcggtatctttttctgggtaactgccaaaccaccacaaatttcaagtttccatttaactcttcaacttcaacccaaccaaatttatttgcttaattgtgcagaaccactccctatatcttctaggtgctttcattcgttccgagtaaaatgcctcaaattggacttgtttctgctgttaacttgagagtccaaggtagttcagcttatctttggagctcgaggtcgtcttctttgggaactgaaagtcgagatggttgcttgcaaaggaattcgttatgttttgctggtagcgaatcaatgggtcataagttaaagattcgtactccccatgccacgaccagaagattggttaaggacttggggcctttaaaggtcgtatgcattgattatccaagaccagagctggacaatacagttaactatttggaggctgcatttttatcatcaacgttccgtgcttctccgcgcccaactaaaccattggagattgttattgctggtgcaggtttgggtggtttgtctacagcaaaatatttggcagatgctggtcacaaaccgatactgctggaggcaagggatgttctaggtggaaaggtagctgcatggaaagatgatgatggagattggtacgagactggtttgcatatattctttggggcttacccaaatattcagaacctgtttggagaattagggattaacgatcgattgcaatggaaggaacattcaatgatatttgcaatgccaagcaagccaggagaattcagccgctttgatttctccgaagctttacccgctcctttaaatggaattttagccatcttaaagaataacgaaatgcttacatggccagagaaagtcaaatttgcaattggactcttgccagcaatgcttggagggcaatcttatgttgaagctcaagatgggataagtgttaaggactggatgagaaagcaaggtgtgccggacagggtgacagatgaggtgttcattgctatgtcaaaggcactcaactttataaaccctgacgaactttcaatgcagtgcattttgatcgcattgaacaggtttcttcaggagaaacatggttcaaaaatggcctttttagatggtaatcctcctgagagactttgcatgccgattgttgaacacattgagtcaaaaggtggccaagtcagactgaactcacgaataaaaaagattgagctgaatgaggatggaagtgtcaagagttttatactgagtgacggtagtgcaatcgagggagatgcttttgtgtttgccgctccagtggatattttcaagcttctattgcctgaagactggaaagagattccatatttccaaaagttggagaagttagtcggagtacctgtgataaatgtacatatatggtttgacagaaaactgaagaacacatatgatcatttgctcttcagcagaagctcactgctcagtgtgtatgctgacatgtctgttacatgtaaggaatattacaaccccaatcagtctatgttggaattggtttttgcacctgcagaagagtggatatctcgcagcgactcagaaattattgatgcaacgatgaaggaactagcaacgctttttcctgatgaaatttcagcagatcaaagcaaagcaaaaatattgaagtaccatgttgtcaaaactccgaggtctgtttataaaactgtgccaggttgtgaaccctgtcggcctttacaaagatccccaatagaggggttttatttagccggtgactacacgaaacagaaatacttggcttcaatggaaggcgctgtcttatcaggaaagctttgtgctcaagctattgtacaggattatgagttacttgttggacgtagccaaaagaagttgtcggaagcaagcgtagtttagctttgtggttattatttagcttctgtacactaaatttatgatgcaagaagcgttgtacacaacatatagaagaagagtgcgaggtgaagcaagtaggagaaatgttaggaaagctcctatacaaaaggatggcatgttgaagattagcatctttttaatcccaagtttaaatataaagcatattttatgtaccactttctttatctggggtttgtaatccctttatatctttatgcaatctttacgttagtt

SEQ ID NO: 28 - DNA, 1749 bp, Capsicum annuum
atgccccaaattggacttgtttctgctgtcaacttgagagtccaaggtaattcagcttatctttggagctcgaggtcttctttgggaactgatagtcaagatggttgctcgcaaaggaattcgttatgttttggtggtagtgactcaatgagtcataggttaaagattcgtaatccccattccataacgagaagattggctaaggatttccggcctttaaaggttgtttgcattgattatccaaggccagagctagacaatacagttaactatttggaggctgcattcttatcatcatcattccgatcttctccgcgcccaaccaaaccactggagattgttattgctggtgcaggtttgggtggtttgtctacagcaaaatatttggcagatgctggtcacaaaccaatactgctggaggcaagggatgttctaggtggaaaggtagctgcatggaaagatgatgatggagattggtatgagactggtttgcacatattctttggggcttacccaaatatgcagaacctatttggagaattagggataaatgatcgattgcaatggaaggaacattcgatgatatttgcaatgccaaacaagccaggagaattcagccgctttgatttccccgaagctttacctgctcctttaaatggaattttggcaatcctaaagaacaatgaaatgcttacatggccagaaaaagtcaaatttgcaattggactcttgccagcaatgcttggtgggcaatcttatgttgaagctcaagacgggataagtgttaaggactggatgagaaaacaaggtgtgccggatagggtgacggatgaggtgttcatcgccatgtcaaaggcacttaacttcataaatcctgatgagctttcgatgcagtgcatcttgatcgcgttgaacagatttcttcaggagaaacatggttcaaaaatggcctttttagatggtaatcctcctgagagactttgcatgccgattgttgaacatatcgagtcaaaaggtggacaagtcagactgaactcacgaataaaaaagattgagctgaatgaggatggaagtgtcaagtgttttatactgaacgatggtagtacaattgagggagatgcttttgtgtttgcgactccagtggatattttcaagcttcttttgcctgaagactggaaagagattccatatttccaaaagttggagaagttagttggagtacctgtgataaatgtccatatatggtttgacagaaaactgaagaacacatctgataatttgctcttcagcagaagcccactgctcagtgtgtatgctgacatgtccgtcacatgtaaggaatattacgaccccaacaagtccatgttggaattggtctttgcgcctgcagaagagtgggtatctcgcagtgactctgaaattattgatgctacaatgaaggaactagcaaagctatttcctgatgaaatttcggcggatcagagcaaagcaaaaatattgaagtatcatgttgtcaaaactccaaggtctgtatataaaactgtgccaggttgtgaaccctgtcggctcttgcaaagatcccctgtagaggggttttatttagctggtgactacacgaaacagaaatacttggcttcaatggaaggtgctgtcttatcaggaaagctttgtgcacaagctattgtacaggattacgagttacttgttggccggagccagaggaagttggcagaaacaagtgtagtttag

SEQ ID NO: 29 - DNA, 2264 bp, Zea mays
ctccaaatgcggaggtctcgactcttctctcttcctccatctttatcatcgccccacgtacacacccaattcctcgcaactgggctcccccgcctccacgacactgccccccgtctcaagtccgccgcctccattcttcagctctcctatcctccgcctagaatatcttcatcggtattttaccaacctggatcaatttactcacgatactctgaagcgtatacatatgccatatgggaaatgacttcatagctgtgggttgtcttatggctccttgaatttgcagtagtctgcctgtacctattggctgaagcagagctgacccccactttatcaagagttgctcaacgatggacactggctgcctgtcatctatgaatattactggagctagccagacaagatcttttgcggggcaacttcctcctcagagatgttttgcgagtagtcactatacaagctttgccgtgaaaaaacttgtctcaaggaataaaggaaggagatcacaccgtagacatcctgccttgcaggttgtctgcaaggattttccaagacctccactagaaagcacaataaactatttggaagctggacagctctcttcattttttagaaacagcgaacgccccagtaagccgttgcaggtcgtggttgctggtgcaggattggctggtctatcaacagcgaagtatctggcagatgctggccataaacccatattgcttgaggcaagagatgttttgggtggaaaggtagctgcttggaaggatgaagatggagattggtacgagactgggcttcatatattttttggagcttatcccaacatacagaatctgtttggcgagcttaggattgaggatcgtttgcagtggaaagaacactctatgatattcgccatgccaaacaagccaggagaattcagccggttcgatttcccagaaactttgccagcacctataaatgggatatgggccatattgagaaacaatgaaatgcttacttggccggagaaggtgaagtttgcaatcggacttctgccagcaatggttggtggtcaaccttatgttgaagctcaagatggcttaaccgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcaaataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttatttcctgatgaaattgctgctgatcagagtaaagcaaagattcttaagtatcatattgtgaagacaccgagatcggtttacaaaactgtcccaaactgtgagccttgccggcctctccaaaggtcacctatcgaaggtttctatctagctggtgattacacaaagcagaaatacctggcttctatggaaggtgcagtcctatccgggaagctttgtgcccagtccatagtgcaggattatagcaggctcgcactcaggagccagaaaagcctacaatcaggagaagttcccgtcccatcttagttgtagttggctttagctatcgtcatccccactgggtgctatcttatctcctatttcaatgggaacccacccaatggtcatgttggagacaacacctgttatggtcctttgaccatctcgtggtgactgtagttgatgtcatattcggatatatatgtaaaaggacctgcatagcaattgttagaccttggaaaaaaa

SEQ ID NO: 30 - DNA, 2027 bp, Oryza sp.
gtttatgacagcatctgccagatattttgcaggacaacttcctactcataggtgcttcgcaagtagcagcatccaagcactgaaaggtagtcagcatgtgagctttggagtgaaatctcttgtcttaaggaataaaggaaaaagattccgtcggaggctcggtgctctacaggttgtttgccaggactttccaagacctccactagaaaacacaataaactttttggaagctggacaactatcctcatttttcagaaacagtgaacaacccactaaaccattacaggtcgtgattgctggagcaggattagctggtttatcaacggcaaaatatctggcagatgctggtcataaacccatattgcttgaggcaagggatgttttgggtggaaagatagctgcttggaaggatgaagatggagattggtatgaaactgggcttcatatcttttttggagcttatcccaacatacagaacttgtttggcgagcttggtattaatgatcggttgcaatggaaggaacactccatgatatttgccatgccaaacaagccaggagaatccagccggtttgattttcctgaaacattgcctgcacccttaaatggaatatgggccatactaagaaacaatgaaatgctaacttggccagagaaggtgaagtttgctcttggacttttgccagcaatggttggtggccaagcttatgttgaagctcaagatggttttactgtttctgagtggatgaaaaagcagggtgttcctgatcgagtgaacgatgaagttttcattgcaatgtcaaaggcacttaatttcataaatcctgatgagttatccatgcagtgcattctgattgctttaaaccgatttcttcaggagaagcatggttctaagatggcattcttggatggtaatcctcctgaaaggttatgcatgcctattgttgaccatgttcgctctttgggtggtgaggttcggctgaattctcgtattcagaaaatagaacttaatcctgatggaacagtgaaacactttgcacttaccgatggaactcaaataactggagatgcttatgtttttgcaacaccagttgatatcttgaagcttcttgtacctcaagagtggaaagaaatatcttatttcaagaagctggagaagttggtgggagttcctgttataaatgttcatatatggtttgatagaaaactgaagaacacatatgaccaccttcttttcagcaggagttcacttttaagtgtttatgcggacatgtcagtaacttgcaaggaatactatgatccaagccgttcaatgctggagttggtctttgctcctgcagaggaatgggttggacggagtgacactgaaatcatcgaagcaactatgcaagagctagccaagctatttcctgatgaaattgctgctgatcagagtaaagcaaagattctgaagtatcatgttgtgaagacaccaagatctgtttacaagactatcccggactgtgaaccttgccgacctctgcaaagatcaccgattgaagggttctatctagctggtgactacacaaagcagaaatatttggcttcgatggagggtgcagttctatctgggaagctttgtgctcagtctgtagtggaggattataaaatgctatctcgtaggagcctgaaaagtctgcagtccgaagttcctgttgcctcctagttgtagtcaggactattcccaatggtgtgtgtgtcatcatcccctagtcagtttttttctatttagtgggtgcccaactctccaccaatttacacatgatggaacttgaaagatgcctattttggtcttatcatatttctgtaaagttgatttgtgactgagagctgatgccgatatgccacgctggagaaaaagaacattatgtaaaacgacctgcatagtaattcttagacttttgcaaaaggcaaaaggggtaaagcgaccttttttttctatgtgaagggattaagagaccttaaaaaaaaaaaaaaaaaaaaaaaaa

SEQ ID NO: 31 - DNA, 1931 bp, Lycopersicon esculentum
ttttgtctttctttcttgttaacccattttcttgatatttaacaagaaaagtttctttcttttttttcctaccctcataattgggtagagaacaattcccatggctacttcttcagcttatctttcttgtcctgcaacttctgctactggaaagaaacatgttttcccaaatgggtcacctggattcttggtttttggtggtacccgtttgtccaaccggttagtgacccgaaagtcggttattcgggctgatttggattctatggtttctgatatgagtaccaacgctccaaaagggctatttccacccgagcctgaacattatcgggggccaaagctgaaagtagctattattggagctgggcttgcaggcatgtcgactgctgtggagctcttggatcaaggacatgaggtggatatatacgaatcaaggacttttattggtgggaaagtgggttcttttgttgatagacgtgggaaccacattgaaatgggactgcacgtgttctttggttgttataataatctgttccgtctgttgaaaaaggtgggtgctgaaaaaaatctgctagtgaaggagcatactcacacatttgtaaataaagggggtgaaataggggaacttgatttccgctttccagttggagcacccttacatggaattaatgcatttctgtctactaatcagttaaagatttatgataaagctagaaatgctgtagctcttgcccttagtccagtggtgcgggctttagttgatccggatggtgcattgcagcagatacgcgatctagataatgtaagcttttctgagtggtttctgtctaaaggtgggacgcgtgctagcatccagaggatgtgggatcctgttgcatatgctcttggattcattgactgtgataacatgagtgctcggtgtatgctcactatatttgcattatttgccacaaaaacagaggcttccctattacgcatgcttaaaggttctcctgacgtttatttgagtggtccaattaagaagtacatcatggacaaagggggcaggttccatctgaggtggggatgcagagaggtactctatgagacgtcctctgatggaagcatgtatgttagtgggcttgccatgtcaaaggccactcagaagaaaattgtaaaagctgatgcatatgtggctgcatgtgatgtccctggaattaaaagattggttcctcagaagtggagggaattggaattctttgacaacatttacaaattggtcggagtgcctgttgttaccgtacaactacgctacaatggctgggttacagagttgcaggacttggagcgttcgaggcaattgaagcgcgctgcaggattggacaatctcctctatacgccagatgcagatttctcttgctttgcagatcttgcattggcatctccagatgattactacattgagggacaaggctcattgcttcaatgtgtccttacacctggtgacccttacatgcctctatcaaatgatgaaatcattaaaagagttacaaagcaggttttggcattatttccttcgtcccaaggtcttgaggttacctggtcatcagttttgaagataggacaatctttatatcgtgaaggacctggtaaagacccattcagacctgatcagaagacgccagtggaaaatttctttcttgctggctcatatacaaaacaggactacatcgatagcatggaaggagcaactctttcaggtaggcaagcttctgcatacatatgtaatgttggagagcagctgatggcgttgcgtaaaaagatcactgctgctgagttgaatgacatctctaaaggtgtgtccctatctgatgagttgagtcttgtctgatgacagactgcaaatcatccaaatacaactcagttaggcatcgcacaaggaagaattcttctaa

SEQ ID NO: 32 - DNA, 1982 bp, Capsicum annuum
cacaattctatggctacttgttcagcttatctttgttgtcctgccacttctgcttctttaaagaaacgtgtttttccagatgggtccgctggattcttgttttttggtggtcgtcgtttgtcgaaccggttagtgaccccaaagtctgtcatccgagctgatttgaactccatggtctctgacatgagtaccaacgctccaaaagggctatttccacctgaacctgaacattatcgggggccaaagctgaaagtagctattattggagctggccttgcaggcatgtcgactgctgtggagctcttggatcaaggacatgaggtggatatatatgaatcaaggaccttcattggtgggaaagtgggttcttttgttgataaacgtgggaaccacattgaaatgggactgcacgtgttctttggttgctataataatctattccgtctgatgaaaaaggtgggtgctgaaaaaaatctgctagtgaaggagcatactcacacatttgtaaataaagggggtgaaataggggagcttgatttccgctttccagttggagcgcccttacatggaattaatgcatttttgtctactaatcaactaaagacttatgataaagctagaaatgctgtagctcttgcccttagtccagtggtgcgggctttagttgatccagatggcgcattgcagcagatacgtgatctagatagtgtaagcttttctgattggtttatgtctaaaggagggacgcgcgctagcatccagaggatgtgggatcctgttgcatatgctcttggattcattgactgtgacaatatcagtgctcggtgtatgctcactatatttgcattatttgccactaaaacggaggcttccctactgcgcatgcttaaaggttctcctgacgtttatttgagtggtccaattaagaagtacatcatagacaaggggggaaggttccatctgaggtggggatgcagagaggtactctacgagacatcctctgatggaagcatgtatgttagcgggcttgccatgtcaaaggccactcagaagaaaattgtaaaagctgatgcctatgttgccgcatgtgtagtacctggaattaaaagattagtacctcagaagtggagggaattggaattctttggcaacatttacaaactgattggagtgcctgttgttactgtgcaactacgatacaatggctgggttacggagttgcaggacttggagcgttcaaggcaatcaaagcgcgctacaggtttggacaatctcctgtacacgccagatgcagatttctcttgttttgcagaccttgcattggcatctccagaagattattacattgagggacaaggctcgttgcttcaatgtgtccttacgcctggcgacccttacatgcctctaccaaatgaagaaatcataagaagagtgtcaaagcaggttttggcgttatttccttcttcccaaggtcttgaggtaacctggtcatcagttgtgaagattgggcaatccttatatcgtgaaggacctggtaaagacccgttcagacctgatcaaaagacgccagtggaaaatttctttcttgctggctcatatacaaaacaggactacatcgatagtatggaaggggcaactctttcaggcagacaagcttctgcatacatatgtgatgctggagagcagctgttggcgctgcgaaaaaagattgctgctgctgagttaaacgagatctctaaaggtgtatcgctatcggatgagttgagtcttgtctgatgactgcaaatcattcagaaatataattcagttaggcagtgcataaggaagaattcttctaaatttttgagtctcacaattatggaaatcaaaatatgttttaaaaatgttgtatgtatgtaatattagtaaatcttcatagtgatgtatgtatctattctgccacgcttcagtttagtgaaatggaacttattgctgcatcaatc

SEQ ID NO: 33 - DNA, 2265 bp, Zea mays
ccctgccacgacgcccgcgacaaatccctgcgcgacggcatcttcgcctcccatcccctcccagcttcccctcccactccggccctcacacaaattgcccctcttcttctcctcctctttacacgctgccgaccacggctgccgccaaccacccgccccacccgtccaccgctgccgagtgctagccatttggagctgccgcgccatggcgtccgtggccgccaccaccacgctggcaccggcactcgccccgcgccgggcgcggccagggactgggctcgtgccgccgcgccgggcctcggccgtcgctgctcgctcgaccgtaacgtctccgacatggcgtcaacgctcccaaaggttattcccacccgagccagagcactacaggggcccgaagctcaaggtggccatcataggggcaggccttgcgggcatgtccaccgctgttgagctcttggaccagggccatgaggttgatttgtacgagtcccgtccgtttatcggtggcaaggttggctcctttgttgacaggcaaggaaaccatatcgagatggggctgcatgtgttcttcgggtgctacagcaatctcttccgcctcatgaagaaggttggcgctgataataatctgctggtgaaggaacatacccatacttttgtaaataaagggggcacgattggtgaacttgattttcggttcccggtgggagctccgttacatggcattcaagcattcctaagaactaatcagctcaaggtttatgataaagcaagaaatgcagttgctcttgcccttagtccagttgttcgggctctggttgatcctgatggtgcattgcagcaagtgcgggacttggatgatataagtttcagtgattggttcatgtccaaagggggtactcgggagagtatcacaagaatgtgggatcctgttcgttacgctttgggtttcattgactgtgataatatcagtgcacgttgcatgcttactattttcaccttgtttgccacaaagacagaggcatccctgttacgcatgttaaagggttcacctgatgtttacttaagtggtccaataaagaagtatataacagacaggggtggtaggtttcacttaaggtggggatgcagagaggttctctatgagaagtcacctgatggagagacctatgttaagggccttctactcaccaaggctacaagtagagagataatcaaagctgatgcatacgtcgcagcctgtgatgttccaggtatcaaaagattacttccatcagaatggagggagtgggaaatgtttgacaatatctacaagttagatggtgtccctgttgtcactgtccagctccgctacaacggatgggtcactgaacttcaagatttggagaaatcaagacaactgcaaagggcggttgggttggataaccttttgtacacggcggatgcagacttttcctgtttttcggaccttgctctctcatctcctgctgattactacattgaagggcaaggttccctgatccaagctgtgctgactcctggagatccatacatgccattgccaaacgaggagatcattagtaaggttcaaaagcaggttgtagaactgttcccatcttcccggggcttagaagttacatggtccagtgtggtaaagatcggacaatcgctgtaccgtgaggctcctggaaacgacccattcaggcctgatcagaagacgcccgttaaaaacttcttcctctctggatcttacacgaaacaggactacatcgacagcatggaaggagcaactctctccggcaggcgaacgtcggcctacatctgcggtgccggggaggagctgctggccctccgaaagaagctactcatcgacgacggcgagaaggcgctggggaacgttcaagtcctgcaggctagctgaacaacccctcctgcactgcagagaagcttggatctttccaaccacacatacatgctggaatggacaaaccaaccaaccattgtcttttctcgcttcagggtgctggcgattcccgcagcaacctcctgtgtatcgtatccaatttgagcattagatctgccccccccccctgcaggcgtttctttcctatccctgatccgagaagcagggtgtagtctaggtggctggcatacgggattacatcaggcagtgtgtaagttcagctggaactcgattggtaattgggatggatgattgatgatatatatatagcacacactgttcttgcgtcttgcaaaaaaaaaaaa

SEQ ID NO: 34 - DNA, 2472 bp, Oryza sp.
ccctgccacgacgcccgcgacaaatccctgcgcgacggcatcttcgcctcccatcccctcccagcttcccctcccactccggccctcacacaaattgcccctcttcttctcctcctctttacacgctgccgaccacggctgccgccaaccacccgccccacccgtccaccgctgccgagtgctagccatttggagctgccgcgccatggcgtccgtggccgccaccaccacgctggcaccggcactcgccccgcgccgggcgcggccagggactgggctcgtgccgccgcgccgggcctcggccgtcgctgctcgctcgaccgtaacgtctccgacatggcgtcaacgctcccaaaggttattcccacccgagccagagcactacaggggcccgaagctcaaggtggccatcataggggcaggccttgcgggcatgtccaccgctgttgagctcttggaccagggccatgaggttgatttgtacgagtcccgtccgtttatcggtggcaaggttggctcctttgttgacaggcaaggaaaccatatcgagatggggctgcatgtgttcttcgggtgctacagcaatctcttccgcctcatgaagaaggttggcgctgataataatctgctggtgaaggaacatacccatacttttgtaaataaagggggcacgattggtgaacttgattttcggttcccggtgggagctccgttacatggcattcaagcattcctaagaactaatcagctcaaggtttatgataaagcaagaaatgcagttgctcttgcccttagtccagttgttcgggctctggttgatcctgatggtgcattgcagcacccacgcgtccgcccacgcgtccggattggtgaacttgattttcggtttcctgtgggagctccgttacatggtatccaagcattcctacgaactaaccaactcaaggtttatgataaagcaagaaatgccgttgctcttgctctaagcccagttgttcgagctcttgttgatccagatggtgcattgcagcaagtacgggatttggatgatgtaagtttcagcgattggttcttgtcgaaaggtggtactcgagagagcatcacaaggatgtgggatcctgttgcctatgctcttggtttcattgactgtgataatatcagtgcacgttgcatgcttaccattttcactctgtttgccacaaaaacagaggcatctttattacgcatgctaaagggttcacctgatgtttatctgagtggtccaataaagaagtacataacagacaggggtggtaggtttcacctgaggtggggatgtagggaggttctctatgataagtcacctgatggggaaacctatgttaaaggccttctcctatccaaggctacaagtagagagataatcaaagcagatgcatatgtcgcagcttgtgatgtcccggggatcaaaagacttttaccttctgaatggaggcaatgggatacatttgacaacatctacaagttagatggtgttcctgtagtcacagtacagcttcgttataatggatgggttacagaacttcaagatttggagaaatcaagacaactgaaaaaggcagttggcttggataatcttctctacactccagatgcagatttttcatgtttttcagaccttgcactttcatctcctgctgactactacattgaaggacaaggttccttgatccaagctgtgctaacccctggcgatccttacatgccattgccgaatgaggagataattagcaaggttcaaaagcaggtcttagaattgttcccgtcatcacaaggcttggaacttacatggtcgagtgtggtgaaaatcggtcaatcattgtaccgcgagtcaccaggaaatgatccatttagacctgatcaaaagacaccagttaaaaacttcttcctgtctggctcttacacaaaacaggactacattgacagcatggaaggggcaactctctcaggcaggagaaccgcggcctacatctgtggtgcaggagaggagctgcttcgccctccgaaagaagctcattgtcgacgacagcggagaaggccaggggtaaggtcgacggcccttcagacaagctgagcttcctcaaatgacacatgctggagtgagtggattgctatgcccaaaacaaaaacagcttcctgggtgtagtaggcgatttccgcagcgactctcatgtaaatcctacttgattgagcatttaggtccaatctgctgctgccctttttgccttgacacgatcgttcgttcgcccgtcaatggtgtgttcttcgttattgtgaatttgtgattgggaaccaaaggtggcatacgggattacatcaggcagcgtgtgttttgttcagcttaaccgatcattgaacccattgatgatgatgatgatgtttatatagtgcacacatcacttaaaaaaaaaaaaaaaaaaaaaaaaaaaaaaa

SEQ ID NO: 35 - DNA, 40 bp, Artificial Sequence
  Primer
cgtcggcctgcatggccctacttctggctatttctcagtg

SEQ ID NO: 36 - DNA, 26 bp, Artificial Sequence
  Primer
ctgtccatggcggccatcacgctcct

SEQ ID NO: 37 - DNA, 40 bp, Artificial Sequence
  Primer
cgatggcctgcatggccctaggtctggccatttctcaatg

SEQ ID NO: 38 - DNA, 32 bp, Artificial Sequence
  Primer
taggataagatagcaaatccatggccatcata